(19) United States
(12) Patent Application Publication
(10) Pub. No.: US 2021/0327553 A1
(43) Pub. Date: Oct. 21, 2021

(54) PREDICTION OF ADVERSE DRUG REACTION BASED ON MACHINE-LEARNED MODELS USING PROTEIN FUNCTION SCORES AND CLINICAL FACTORS

(71) Applicant: Cipherome, Inc., San Jose, CA (US)

(72) Inventors: In Gu LEE, San Jose, CA (US); Min Sang KIM, Sunnyvale, CA (US)

(21) Appl. No.: 17/231,970

(22) Filed: Apr. 15, 2021

Related U.S. Application Data
(60) Provisional application No. 63/012,032, filed on Apr. 17, 2020.

(51) Int. Cl. G16H 20/10; G16H 70/40; G16H 50/20; G16B 30/00; G06F 17/11

(52) U.S. Cl. CPC G16H 20/10; G16H 70/40; G06F 17/11; G16B 30/00; G16H 50/20

(57) ABSTRACT

The present disclosure predicts adverse reaction to drugs based on individual genetic and clinical information. The system receives as an input to the system gene sequence information and clinical information for a subject, and determines one or more scores (e.g., protein function score, clinical factor score, drug safety score) based on that information, where the scores can indicate subject's risk of having the adverse drug reaction. The system provides a representation of the prediction and/or information about the associated phenotype for display on a user interface in a client device (e.g., a physician's device).

### 1. CROSS-REFERENCE TO RELATED APPLICATIONS

This application claims priority to U.S. Provisional Patent Application No. 63/012,032 filed on Apr. 17, 2020, which is incorporated by reference in its entirety.

2. BACKGROUND

An adverse drug reaction (ADR) is an unwanted effect caused by taking medication, that can occur suddenly or develop over time. Serious adverse events can involve death, life-threatening conditions, hospitalization, disability, congenital abnormality and conditions requiring intervention to prevent permanent impairment or damage. For example, warfarin caused bleeding in fifteen to twenty percent of patients and intracranial hemorrhage in one to three percent of patients, ranking in the top ten drugs with serious side effects from the 1990s to 2000s. ADRs have been studied and are associated with or caused by various factors, such as abnormal pharmacokinetics due to genetic factors and comorbid disease states, and interactions between a drug and a disease state or between multiple drugs. Pharmacogenomics and pharmacovigilance studies have linked ADRs to genetic variations that can lead to abnormal drug metabolism.

To avoid or reduce such adverse drug reactions, it is important to assess the risk prior to taking medication. There have been numerous attempts to make the prediction, for example, using Naranjo algorithm, Venulet algorithm and WHO causality term assessment criteria. The approaches are not perfect and cannot provide an accurate prediction for all the subjects. Accordingly, there is a need for a method of predicting ADRs with better sensitivity and specificity.

### 3. SUMMARY

The present disclosure provides a method, system and computer-readable medium for predicting an adverse drug reaction (ADR) of a subject based on individual genome sequence information and clinical information.

The prediction system receives as an input to the system genome sequence information and clinical information for a subject. The system determines one or more scores (e.g., gene sequence variation score, protein function score, clinical factor score, drug safety score) based on that input sequence and clinical information, where the scores can be used to predict an ADR of the subject. The prediction system can provide a representation of the prediction and/or information about the prediction results for display on a user interface, such as a user interface of the prediction system or a user interface on the device of the subject, of family or friends of the subject, of a physician or caregiver of the subject, among others.

A method of prediction drug responses using gene sequence information was described in PCT/KR2014/007685 and U.S. application Ser. No. 14/912,397, which are incorporated by reference in their entireties herein. The present disclosure provides an improved way of predicting drug responses by adding the use of clinical information to the prediction and/or by using a machine learning approach on the retrospective genomic and phenotypic data. Specifically, it provides how to process, combine, and aggregate a plurality of genetic and clinical information to predict risk of the subject to have an adverse drug response. It was found that some genetic and/or clinical factors should be weighted more heavily than other factors in prediction of the adverse drug effect. The machine learning technique allows for determination of what weights to apply in the system. This new approach allows more accurate prediction of drug responses in patients.

The method described herein allows a physician to tailor how to treat a patient and avoid treating the patient using drugs that will be possibly dangerous for the patient. The method can be used to reliably predict a drug response because the method has been adjusted and optimized to better reflect differences between population groups. The method can be also used to identify or select a patient population who can be treated with a drug. For example, the method can be used for clinical trials to identify a patient population who will get a benefit from the use of the drug.

Specifically, the present invention provides a method for treating a subject based on prediction of an adverse reaction to a drug, comprising the steps of: receiving, by a prediction system, clinical information of the subject related to a plurality of clinical factors (cⱼ); for each of the clinical factors (cⱼ), determining, by the prediction system, a clinical factor score (S_{cj}) based on the clinical information; receiving, by a prediction system, individual gene sequence information of the subject; receiving, by the prediction system, information about a plurality of proteins, wherein each of the proteins is related to pharmacokinetics or pharmacodynamics of the drug; for each of the genes (gₖ) encoding the proteins, determining, by the prediction system, a gene sequence variation score (v) of the gene (gₖ) for the subject by using the individual gene sequence information; and calculating, by the prediction system, an individual protein function score (F_{gk}) associated with the protein by using Equation 2, wherein Equation 2 is:

$${F}_{gk} ( {v}_{1} , … , {v}_{{n}_{k}} ) = {( {\prod }_{j = 1}^{{n}_{k}} {v}_{j , k}^{{b}_{j , k}} )}^{\frac{1}{{\sum }_{j = 1}^{{n}_{k}} {b}_{j , k}}}$$

wherein F_{gk} is the individual protein function score of the protein encoded by the gene gₖ, nₖ is the number of sequence variations of the gene gₖ, v_{j,k} is a gene sequence variation score of an j^{th} gene sequence variation, and b_{j,k} is a weighting assigned to the v_{j,k}; and determining, by the prediction system, a drug safety score (DSS) by using Equation 7, wherein Equation 7 is:

$$ln ( \frac{DSS}{1 - DSS} ) = {B}_{0} + {W}_{g 1} {F}_{g 1} + {W}_{g 2} {F}_{g 2} + ⋯ {W}_{gm} {F}_{gm} + {W}_{c 1} {S}_{c 1} + {W}_{c 2} {S}_{c 2} + ⋯ {W}_{cp} {S}_{cp}$$

wherein B₀ is an intercept, W_{gk} is a weighting assigned to each protein function score F_{gk}, W_{ci} is a weighting assigned to each clinical factor score S_{ci}; and treating the subject with the drug, if the DSS compared to a threshold indicates a low risk of the adverse reaction, and treating the subject with an alternative drug, if the DSS compared to a threshold indicates a high risk of the adverse reaction.

In one aspect, the weighting (b_{j,k}) assigned to the gene sequence variation score v_{j,k} is determined by: obtaining training data including a plurality of training instances for a particular protein, each training instance including a predetermined protein function score for the particular protein and a set of gene sequence variation scores for the particular protein, determining a loss function indicating a difference between the predetermined protein function scores and estimated protein function scores, an estimated protein function score for a training data instance generated by applying Equation 2 to the set of gene sequence variation scores for the training data instance, and reducing the loss function to determine the weightings assigned to the gene sequence variation scores.

In one aspect, the weighting (b_{j,k}) assigned to the gene sequence variation score v_{j,k} is 0 for all the gene sequence variation scores.

In one aspect, the weighting (W_{gk}) assigned to the protein function score F_{gk} and the weighting (W_{ci}) assigned to the clinical function score S_{ci} is determined by: obtaining training data including a plurality of training instances including information for a plurality of individuals, each training instance including an actual outcome of whether the individual for the training instance experienced an adverse drug reaction and a set of protein function scores and a set of clinical function scores for the individual, determining a loss function indicating a difference between the actual outcomes and estimated outputs, an estimated output for a training data instance generated by applying Equation 7 to the set of protein function scores and the set of clinical function scores for the training data instance, and reducing the loss function to determine the weightings assigned to the protein function score and the weightings assigned to the clinical function score.

In one aspect, the DSS indicates a low risk of the adverse reaction when the DSS is below a threshold.

In one aspect, the threshold is 0.3, 0.4, or 0.5.

In one aspect, the clinical factors are selected from the group consisting of age, weight, height, sex, ethnicity, concomitant medication, smoking history, alcohol consumption, and lab data.

In one aspect, the gene sequence variation score v_{j,k} calculated using one or more algorithms selected from the group consisting of: SIFT (Sorting Intolerant From Tolerant), PolyPhen (Polymorphism Phenotyping), PolyPhen-2, MAPP (Multivariate Analysis of Protein Polymorphism), Logre (Log R Pfam E-value), MutationAssessor, MutationTaster, MutationTaster2, PROVEAN (Protein Variation Effect Analyzer), PMut, Condel, GERP (Genomic Evolutionary Rate Profiling), GERP++, CEO (Combinatorial Entropy Optimization), SNPeffect, fathmm, CADD (Combined Annotation-Dependent Depletion), and ADME-optimized algorithm.

In one aspect, the gene sequence variation score v_{j,k} is determined using experimental data.

Specifically, the present disclosure also provides a method for treating a subject based on prediction of an adverse reaction to a drug, comprising the steps of: receiving, by a prediction system, individual gene sequence information of the subject; receiving, by the prediction system, information about a protein, wherein the protein is related to pharmacokinetics or pharmacodynamics of the drug, and a gene (g) encoding the protein; determining, by the prediction system, a gene sequence variation score (v) of the gene (g) for the subject by using the individual gene sequence information; calculating, by the prediction system, an individual protein function score associated with the protein by using Equation 2, wherein Equation 2 is:

$${F}_{g} ( {v}_{1} , … , {v}_{n} ) = {( {\prod }_{i = 1}^{n} {v}_{i}^{{b}_{i}} )}^{\frac{1}{{\sum }_{i = 1}^{n} {b}_{i}}}$$

wherein Fg is the individual protein function score of the protein encoded by the gene g, n is the number of sequence variations of the gene g, vᵢ is a gene sequence variation score of an i^{th} gene sequence variation, and bᵢ is a weighting assigned to the gene sequence variation score vᵢ of the i^{th} gene sequence variation, and wherein the weighting (bᵢ) assigned to the gene sequence variation score vᵢ is determined by: obtaining training data including a plurality of training instances for a particular protein, each training instance including a predetermined protein function score for the particular protein and a set of gene sequence variation scores for the particular protein, determining a loss function indicating a difference between the predetermined protein function scores and estimated protein function scores, an estimated protein function score for a training data instance generated by applying Equation 2 to the set of gene sequence variation scores for the training data instance, and reducing the loss function to determine the weightings assigned to the gene sequence variation scores; predicting, by the prediction system, likelihood of the adverse reaction to the drug based on the individual protein function score compared to a threshold; and treating the subject with the drug, if the prediction step indicates low likelihood of the adverse reaction, and treating the subject with an alternative drug, if the prediction step indicates high likelihood of the adverse reaction.

In one aspect, the gene sequence variation score vᵢ calculated using one or more algorithms selected from the group consisting of: SIFT (Sorting Intolerant From Tolerant), PolyPhen (Polymorphism Phenotyping), PolyPhen-2, MAPP (Multivariate Analysis of Protein Polymorphism), Logre (Log R Pfam E-value), MutationAssessor, MutationTaster, MutationTaster2, PROVEAN (Protein Variation Effect Analyzer), PMut, Condel, GERP (Genomic Evolutionary Rate Profiling), GERP++, CEO (Combinatorial Entropy Optimization), SNPeffect, fathmm, CADD (Combined Annotation-Dependent Depletion), and ADME-optimized algorithm.

In one aspect, the gene sequence variation score vᵢ is determined using experimental data.

In one aspect, the method further comprise the step of: providing, by the prediction system, the drug safety score (DSS) or information related to the predicted adverse reaction to the drug.

The present disclosure also provides a system for predicting an adverse drug reaction of a subject to a drug, the system comprising: a processor; a computer readable storage medium for storing modules executable by a processor, the modules comprising: a communication module configured to receive clinical information of the subject related to a plurality of clinical factors (cⱼ), individual gene sequence information for the subject and a plurality of proteins, wherein each of the proteins is related to pharmacokinetics or pharmacodynamics of the drug; an analysis module configured to: determine a clinical factor score (S_{cj}) for each of the clinical factors (cⱼ), determine a gene sequence variation score (v) of each of the genes (gₖ) encoding the proteins related to pharmacokinetics or pharmacodynamics of the drug, calculate an individual protein function score (F_{gk}) associated with the protein by using Equation 2, wherein Equation 2 is:

$${F}_{gk} ( {v}_{1} , … , {v}_{{n}_{k}} ) = {( {\prod }_{j = 1}^{{n}_{k}} {v}_{j , k}^{{b}_{j , k}} )}^{\frac{1}{{\sum }_{j = 1}^{{n}_{k}} {b}_{j , k}}}$$

wherein F_{gk}is the individual protein function score of the protein encoded by the gene gₖ, nₖ is the number of sequence variations of the gene gₖ, v_{j,k} is a gene sequence variation score of an j^{th} gene sequence variation, and b_{j,k} is a weighting assigned to the v_{j,k}, determine a drug safety score (DSS) by using Equation 7, wherein Equation 7 is:

$$ln ( \frac{DSS}{1 - DSS} ) = {B}_{0} + {W}_{g 1} {F}_{g 1} + {W}_{g 2} {F}_{g 2} + ⋯ {W}_{gm} {F}_{gm} + {W}_{c 1} {S}_{c 1} + {W}_{c 2} {S}_{c 2} + ⋯ {W}_{cp} {S}_{cp}$$

wherein B₀ is an intercept, W_{gk} is a weighting assigned to each protein function score F_{gk}, W_{ci} is a weighting assigned to each clinical factor score S_{ci}, and predict the adverse drug reaction of the subject using the drug safety score (DSS); and an interface generation module configured to provide for display in a user interface on a client device a representation of the prediction for use in treatment of the subject.

In one aspect, the weighting (b_{j,k}) assigned to the gene sequence variation score v_{j,k} is determined by obtaining training data including a plurality of training instances for a particular protein, each training instance including a predetermined protein function score for the particular protein and a set of gene sequence variation scores for the particular protein, determining a loss function indicating a difference between the predetermined protein function scores and estimated protein function scores, an estimated protein function score for a training data instance generated by applying Equation 2 to the set of gene sequence variation scores for the training data instance, and reducing the loss function to determine the weightings assigned to the gene sequence variation scores.

In one aspect, the weighting (b_{j,k}) assigned to the gene sequence variation score v_{j,k} is 0 for all the gene sequence variation scores.

In one aspect, the weighting (W_{gk}) assigned to the protein function score F_{gk} and the weighting (W_{ci}) assigned to the clinical function score S_{ci} is determined by: obtaining training data including a plurality of training instances including information for a plurality of individuals, each training instance including an actual outcome of whether the individual for the training instance experienced an adverse drug reaction and a set of protein function scores and a set of clinical function scores for the individual, determining a loss function indicating a difference between the actual outcomes and estimated outputs, an estimated output for a training data instance generated by applying Equation 5 to the set of protein function scores and the set of clinical function scores for the training data instance, and reducing the loss function to determine the weightings assigned to the protein function score and the weightings assigned to the clinical function score.

In one aspect, the DSS indicates a low likelihood of the adverse reaction when the DSS is below a threshold.

In one aspect, the threshold is 0.3, 0.4, or 0.5.

In one aspect, the clinical factors are selected from the group consisting of age, weight, height, sex, ethnicity, concomitant medication, smoking history, alcohol consumption, and lab data.

In one aspect, the gene sequence variation score v_{j,k} is calculated using one or more algorithms selected from the group consisting of: SIFT (Sorting Intolerant From Tolerant), PolyPhen (Polymorphism Phenotyping), PolyPhen-2, MAPP (Multivariate Analysis of Protein Polymorphism), Logre (Log R Pfam E-value), MutationAssessor, MutationTaster, MutationTaster2, PROVEAN (Protein Variation Effect Analyzer), PMut, Condel, GERP (Genomic Evolutionary Rate Profiling), GERP++, CEO (Combinatorial Entropy Optimization), SNPeffect, fathmm, CADD (Combined Annotation-Dependent Depletion), and ADME-optimized algorithm.

In one aspect, the gene sequence variation score v_{j,k} is determined using experimental data.

The present disclosure also provides a system for predicting an adverse drug reaction of a subject to a drug, the system comprising: a processor; a computer readable storage medium for storing modules executable by a processor, the modules comprising: a communication module configured to receive information about a protein, wherein the protein is related to pharmacokinetics or pharmacodynamics of the drug, and a gene (g) encoding the protein; an analysis module configured to: determine a gene sequence variation score (v) of each of the genes (g) for the subject by using the individual gene sequence information, calculate an individual protein function score associated with the protein by using Equation 2, wherein Equation 2 is:

$${F}_{g} ( {v}_{1} , … , {v}_{n} ) = {( {\prod }_{i = 1}^{n} {v}_{i}^{{b}_{i}} )}^{\frac{1}{{\sum }_{i = 1}^{n} {b}_{i}}}$$

wherein Fg is the individual protein function score of the protein encoded by the gene g, n is the number of sequence variations of the gene g, vᵢ is a gene sequence variation score of an i^{th} gene sequence variation, and bᵢ is a weighting assigned to the gene sequence variation score vᵢ of the i^{th} gene sequence variation, wherein the weighting (bᵢ) assigned to the gene sequence variation score vᵢ is determined by: obtaining training data including a plurality of training instances for a particular protein, each training instance including a predetermined protein function score for the particular protein and a set of gene sequence variation scores for the particular protein, determining a loss function indicating a difference between the predetermined protein function scores and estimated protein function scores, an estimated protein function score for a training data instance generated by applying Equation 2 to the set of gene sequence variation scores for the training data instance, and reducing the loss function to determine the weightings assigned to the gene sequence variation scores, and predict the adverse reaction to the drug based on the individual protein function score; and an interface generation module configured to provide for display in a user interface on a client device a representation of the prediction for use in treatment of the subject.

The present disclosure also provides a computer-readable medium comprising an execution module for executing a processor that performs an operation of predicting an adverse reaction of a subject to a drug, comprising the steps of: receiving clinical information of the subject related to a plurality of clinical factors (cⱼ); for each of the clinical factors (cⱼ), determining a clinical factor score (S_{cj}) based on the clinical information; receiving individual gene sequence information of the subject; receiving information about a plurality of proteins, wherein each of the proteins is related to pharmacokinetics or pharmacodynamics of the drug; for each of the genes (gₖ) encoding the proteins, determining a gene sequence variation score (v) of the gene (gₖ) for the subject by using the individual gene sequence information; and calculating an individual protein function score (F_{gk}) associated with the protein by using Equation 2, wherein Equation 2 is:

$${F}_{gk} ( {v}_{1} , … , {v}_{{n}_{k}} ) = {( {\prod }_{j = 1}^{{n}_{k}} {v}_{j , k}^{{b}_{j , k}} )}^{\frac{1}{{\sum }_{j = 1}^{{n}_{k}} {b}_{j , k}}}$$

wherein F_{gk} is the individual protein function score of the protein encoded by the gene gₖ, nₖ is the number of sequence variations of the gene gₖ, v_{j,k} is a gene sequence variation score of an f^{th} gene sequence variation, and b_{j,k} is a weighting assigned to the v_{j,k}; and determining a drug safety score (DSS) by using Equation 7, wherein Equation 7 is:

$$ln ( \frac{DSS}{1 - DSS} ) = {B}_{0} + {W}_{g 1} {F}_{g 1} + {W}_{g 2} {F}_{g 2} + ⋯ {W}_{gm} {F}_{gm} + {W}_{c 1} {S}_{c 1} + {W}_{c 2} {S}_{c 2} + ⋯ {W}_{cp} {S}_{cp}$$

wherein B₀ is an intercept, W_{gk} is a weighting assigned to each protein function score F_{gk}, W_{Ci} is a weighting assigned to each clinical factor score S_{ci}; and predict the adverse drug reaction of the subject using the drug safety score (DSS); and providing for display in a user interface on a client device a representation of the prediction for use in treatment of the subject.

The present disclosure also provides a computer-readable medium comprising an execution module for executing a processor that performs an operation of predicting an adverse reaction of a subject to a drug, comprising the steps of: receiving individual gene sequence information of the subject; receiving information about a protein, wherein the protein is related to pharmacokinetics or pharmacodynamics of the drug, and a gene (g) encoding the protein; determining a gene sequence variation score (v) of the gene (g) for the subject by using the individual gene sequence information; calculating an individual protein function score associated with the protein by using Equation 2, wherein Equation 2 is:

$${F}_{g} ( {v}_{1} , … , {v}_{n} ) = {( {\prod }_{i = 1}^{n} {v}_{i}^{{b}_{i}} )}^{\frac{1}{{\sum }_{i = 1}^{n} {b}_{i}}}$$

wherein Fg is the individual protein function score of the protein encoded by the gene g, n is the number of sequence variations of the gene g, vᵢ is a gene sequence variation score of an i^{th} gene sequence variation, and bᵢ is a weighting assigned to the gene sequence variation score vᵢ of the i^{th} gene sequence variation, and wherein the weighting (bᵢ) assigned to the gene sequence variation score vᵢ is determined by: obtaining training data including a plurality of training instances for a particular protein, each training instance including a predetermined protein function score for the particular protein and a set of gene sequence variation scores for the particular protein, determining a loss function indicating a difference between the predetermined protein function scores and estimated protein function scores, an estimated protein function score for a training data instance generated by applying Equation 2 to the set of gene sequence variation scores for the training data instance, and reducing the loss function to determine the weightings assigned to the gene sequence variation scores; predicting, by the prediction system, the adverse reaction to the drug based on the individual protein function score; and providing for display in a user interface on a client device a representation of the prediction for use in treatment of the subject.

The present disclosure also provides a method for selecting a treatment population from a plurality of subjects for treatment with a drug, comprising the steps of: for each subject in the plurality of subjects: receiving, by a prediction system, clinical information of the subject related to a plurality of clinical factors (cⱼ); for each of the clinical factors (cⱼ), determining, by the prediction system, a clinical factor score (S_{cj}) based on the clinical information of the subject; receiving, by the prediction system, individual gene sequence information of the subject and information about a plurality of proteins, wherein each of the proteins is related to pharmacokinetics or pharmacodynamics of the drug; for each of the genes (gₖ) encoding the plurality of proteins, determining, by the prediction system, a gene sequence variation score (v_{j,k}) for each of a gene sequence variation of the gene (gₖ) for the subject by using the individual gene sequence information; and calculating, by the prediction system, an individual protein function score (F_{gk}) associated with the protein by using Equation 2, wherein Equation 2 is:

$${F}_{gk} ( {v}_{1} , … , {v}_{{n}_{k}} ) = {( {\prod }_{j = 1}^{{n}_{k}} {v}_{j , k}^{{b}_{j , k}} )}^{\frac{1}{{\sum }_{j = 1}^{{n}_{k}} {b}_{j , k}}}$$

wherein F_{gk} is the individual protein function score of the protein encoded by the gene gₖ, nₖ is the number of sequence variations of the gene gₖ, v_{j,k} is a gene sequence variation score of anf^{h} gene sequence variation for the gene gₖ, and b_{j,k} is a weighting assigned to the v_{j,k}; and determining, by the prediction system, a drug safety score (DSS) for the subject by using Equation 7, wherein Equation 7 is:

$$ln ( \frac{DSS}{1 - DSS} ) = {B}_{0} + {W}_{g 1} {F}_{g 1} + {W}_{g 2} {F}_{g 2} + ⋯ {W}_{gm} {F}_{gm} + {W}_{c 1} {S}_{c 1} + {W}_{c 2} {S}_{c 2} + ⋯ {W}_{cp} {S}_{cp}$$

wherein B₀ is an intercept, W_{gk} is a weighting assigned to each protein function score F_{gk}, W_{ci} is a weighting assigned to each clinical factor score S_{ci}; and selecting a treatment population from the plurality of subjects for treatment with the drug based on the determined DSS for the plurality of subjects, the DSS of the selected treatment population indicating a low risk of an adverse reaction to the drug.

In one aspect, selecting the treatment population from the plurality of subjects comprises selecting the treatment population for a clinical study of the drug.

In one aspect, the weighting (b_{j,k}) assigned to the gene sequence variation score v_{j,k} is determined by: obtaining training data including a plurality of training instances for a particular protein, each training instance including a predetermined protein function score for the particular protein and a set of gene sequence variation scores for the particular protein, determining a loss function indicating a difference between the predetermined protein function scores and estimated protein function scores, an estimated protein function score for a training data instance generated by applying Equation 2 to the set of gene sequence variation scores for the training data instance, and reducing the loss function to determine the weightings assigned to the gene sequence variation scores.

In one aspect, the weighting (b_{j,k}) assigned to the gene sequence variation score v_{j,k} is 0 for all the gene sequence variation scores.

In one aspect, the weighting (W_{gk}) assigned to the protein function score F_{gk} and the weighting (W_{ci}) assigned to the clinical function score S_{ci} is determined by: obtaining training data including a plurality of training instances including information for a plurality of individuals, each training instance including an actual outcome of whether the individual for the training instance experienced an adverse drug reaction and a set of protein function scores and a set of clinical function scores for the individual, determining a loss function indicating a difference between the actual outcomes and estimated outputs, an estimated output for a training data instance generated by applying Equation 7 to the set of protein function scores and the set of clinical function scores for the training data instance, and reducing the loss function to determine the weightings assigned to the protein function score and the weightings assigned to the clinical function score.

In one aspect, the DSS indicates a low risk of the adverse reaction when the DSS is below a threshold.

In one aspect, the threshold is 0.3, 0.4, or 0.5.

In one aspect, the clinical factors are selected from the group consisting of age, weight, height, sex, ethnicity, concomitant medication, smoking history, alcohol consumption, and lab data.

In one aspect, the gene sequence variation score v_{j,k} is calculated using one or more algorithms selected from the group consisting of: SIFT (Sorting Intolerant From Tolerant), PolyPhen (Polymorphism Phenotyping), PolyPhen-2, MAPP (Multivariate Analysis of Protein Polymorphism), Logre (Log R Pfam E-value), MutationAssessor, MutationTaster, MutationTaster2, PROVEAN (Protein Variation Effect Analyzer), PMut, Condel, GERP (Genomic Evolutionary Rate Profiling), GERP++, CEO (Combinatorial Entropy Optimization), SNPeffect, fathmm, CADD (Combined Annotation-Dependent Depletion), and ADME-optimized algorithm.

In one aspect, the gene sequence variation score v_{j,k} is determined using experimental data.

In one aspect, the method further comprises the step of obtaining a curve representing the DSS for the plurality of subjects.

In one aspect, the method further comprises the step of determining an area under the curve (AUC), a standardized area under the curve (S-AUC), an area upper the curve (AUPC), or a standardized area upper the curve (S-AUPC).

In one aspect, the method further comprises the step of identifying individuals having a DSS below or above a threshold value.

In one aspect, the threshold value (T) is calculated by the Equation:

$$T = μ - κ \sqrt{\frac{1}{n} {\sum }_{i = 1}^{n} {( {DDS}_{i} - μ )}^{2}} ,$$

wherein T is a rational number satisfying 0<T<1, DDSᵢ is an individual drug safety score of an i-th individual (from 1 to n) within the population, n is the number of individuals within the population, κ is a non-zero rational number, and μ is either (i) a mean of the set of individual drug safety scores or (ii) an area under the curve of the set of individual drug safety scores.

In one aspect, the threshold value (T) is determined based on the shape of the curve.

In one aspect, the threshold value (T) is calculated based on the change in the slope of the curve.

In one aspect, the threshold value (T) is determined by comparing the curve with a different curve corresponding to a different drug having similar pharmacodynamics or pharmacokinetics or a different drug previously identified to be unsafe.

In one aspect, the threshold value (T) ranges from 0.1 to 0.5, from 0.2 to 0.4, or from 0.25 to 0.35, or is 0.3.

In one aspect, the method further comprises the step of providing a list of the individuals having a drug safety score below the threshold value or above the threshold value.

### 4. BRIEF DESCRIPTION OF THE DRAWINGS

FIG. 1A illustrates an overall system environment for providing information related to adverse drug reaction generated based on gene sequence information and clinical information by a prediction system, in accordance with an embodiment.

FIG. 1B provides a flowchart summarizing an exemplary method of predicting adverse drug reaction and providing the information for treatment of a patient.

FIG. 1C provides a flowchart summarizing an exemplary method of using drug safety score and prediction of adverse drug reaction.

FIG. 1D provides a flowchart summarizing an exemplary method of using the prediction system of the present invention to obtain information related to adverse drug reaction.

FIG. 2 provides demographic information of population subject to the study described in Example 1.

FIG. 3A-3D provide AUROC curve of step-wise multiple logistic regression analysis results. Step-wise multiple logistic regression was performed using combination of variables, genotype of CYP2C9 and VKORC1 (rs9923231) only (FIG. 3A), protein function scores of eleven warfarin-associated genes (FIG. 3B), known variables in warfarin dosing calculator (FIG. 3C), protein function scores of eleven warfarin-associated genes and variables in warfarin-dosing calculator (FIG. 3D).

FIG. 4 shows the step-wise multiple logistic regression model obtained from the study described in Example 1.

FIG. 5A shows the values and statistical characteristics of weightings determined for a stepwise logistic regression model using 6 protein function scores to the study described in Example 2. FIG. 5B provides AUROC curves of step-wise multiple logistic regression models using protein function scores of the 6 chloroquine-associated genes.

FIG. 6A-6F provide the AUROC curves of step-wise multiple logistic regression models. Step-wise multiple logistic regression was performed using demographic information (FIG. 6A), drug-drug-interaction (DDI) factors (FIG. 6B), protein function scores of 6 chloroquine-associated genes (FIG. 6C), combination of demographic information and the protein function scores of 6 chloroquine-associated genes (FIG. 6D), combination of DDI factors and the protein function scores of chloroquine-associated genes (FIG. 6E), and combination of demographic information, DDI factors, and the protein function scores of 6 chloroquine-associated genes (FIG. 6F).

FIG. 7 shows the AUC distribution using 6 random genes for the study described in Example 2.

FIG. 8A-8D provide the AUROC curves of step-wise multiple logistic regression models obtained from the study described in Example 3. Step-wise multiple logistic regression was performed using demographic information and protein function scores of DOAC-related genes (FIGS. 8A-8B), demographic information, protein function scores, and drug-drug-interaction (DDI) factors (FIG. 8C), and demographic information, protein function scores, and HASBLED factors (FIG. 8D). The figures depict various embodiments of the present invention for purposes of illustration only. One skilled in the art will readily recognize from the following discussion that alternative embodiments of the structures and methods illustrated herein may be employed without departing from the principles of the invention described herein.

### 5. DETAILED DESCRIPTION

### 5.1. DEFINITIONS

Unless defined otherwise, all technical and scientific terms used herein have the meaning commonly understood by a person skilled in the art to which this invention belongs. As used herein, the following terms have the meanings ascribed to them below.

The term “adverse drug reaction” or “ADR” as used herein refers to an unwanted, undesirable effect caused by taking a drug. Adverse drug reactions include a serious adverse event involving death, life-threatening conditions, hospitalization, disability, congenital abnormality and conditions requiring intervention to prevent permanent impairment or damage, but are not limited thereto. The adverse drug reaction can be less serious forms. The term can refer to a particular adverse symptom caused by taking a drug, e.g., bleeding, an immune response, pain, damage to a tissue, etc. or a combination thereof.

The term “gene sequence variation information” used herein means information about a substitution, addition, or deletion of a base constituting a genomic sequence of a gene. The base can be in a coding region (e.g., exon) or in a non-coding region (e.g., intron, promoter, or other regulatory sequence). Such substitution, addition, or deletion of the base may result from various causes, for example, mutation, breakage, deletion, duplication, inversion, and/or translocation of a chromosome or portion of a chromosome. Individual gene sequence variation information refers to gene sequence variation information of a particular individual or subject.

The term “gene sequence variation score” used herein refers to a numerical score of a degree of the individual genome sequence variation that causes an amino acid sequence variation (substitution, addition, or deletion) of a protein encoded by a gene or a transcription control variation and thus causes a significant change or damage to a structure and/or function of the protein. The gene sequence variation score can be calculated considering various factors including a degree of evolutionary conservation of amino acid in a genome sequence, and a degree of an impact of a modified amino acid on a structure or function of the corresponding protein. The gene sequence variation score can be calculated computationally or based on experimental data representing relationship between a modified amino acid and function of the corresponding protein.

The terms “protein function score”, “gene function score” or “GFS” used herein refer to a numerical score calculated by summarizing selected gene sequence variation scores, each corresponding to a variation found in a gene encoding a single protein. Some or all gene sequence variation scores are selected based on their relevancy to the protein function or their absolute or relative values. The protein function score is related to a phenotype of a protein encoded by the gene, for example, functional deficiency or activity level of the protein. Individual protein function score refers to a protein score of a particular individual or subject.

The term “clinical factor score” used herein refers to a numerical score representing various clinical factors such as patient medical history, biographical information about the patient (age, gender, height, weight, BMI, race, ethnicity, smoking history, alcohol consumption, etc.), vital sign data and history (blood pressure, heart rate, temperature, oxygen level, etc.), lab data (hemoglobin level, international normalized ratio (INR), serum albumin, AST/ALT ratio, etc.) data about past medical treatments and conditions of the patient, current symptoms, prior diagnoses or prognoses, medical images taken of the patient, drug or treatments currently or previously used, concurrent medication, adherence by the patient to treatments, etc. The clinical factor score can represent a unit number (e.g., a measured value such as height or weight) or as a number indicating a relevant category (e.g., smoking can be presented by units of pack-per-year or pack-per-day or categorically such as ex-smoker, occasional smoker, regular smoker, etc.).

The terms “pharmacokinetics,” “pk,” or “pharmacokinetic parameters” used herein refer to characteristics of a drug related to absorption, metabolism, migration, distribution, conversion, and excretion of the drug in the body for a predetermined time period, and includes a volume of distribution (Vd), a clearance rate (CL), bioavailability (F) and absorption rate coefficient (ka) of a drug, or a maximum plasma concentration (Cmax), a time point of maximum plasma concentration (Tmax), an area under the curve (AUC) regarding a change in plasma concentration for a certain time period, and so on.

The terms “pharmacodynamics,” “pd,” or “pharmacodynamic parameters” used herein refer to characteristics involved in physiological and biochemical behaviors of a drug within a body and mechanisms thereof, i.e., responses or effects in the body caused by the drug.

The term “subject” used herein refers to a human or an animal whose gene sequence information is provided to a prediction system for analysis by methods provided herein. A subject can be a patient or a non-patient.

Lists of genes involved in the pharmacodynamics or pharmacokinetics of a predetermined drug or drug group are provided in WO2015026135, US Publication No. 20160210401, WO2016133373, WO2016133374, and WO2016133375, which are incorporated by reference herein in their entireties. Various embodiments of the present invention can be applied to predict phenotypes associated with the genes disclosed in the references.

### 5.2. OTHER INTERPRETATIONAL CONVENTIONS

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50, and any fractional value in between these numbers.

Unless otherwise indicated, reference to a compound that has one or more stereocenters intends each stereoisomer, and all combinations of stereoisomers, thereof.

5.3. SYSTEM FOR PREDICTION OF ADRS

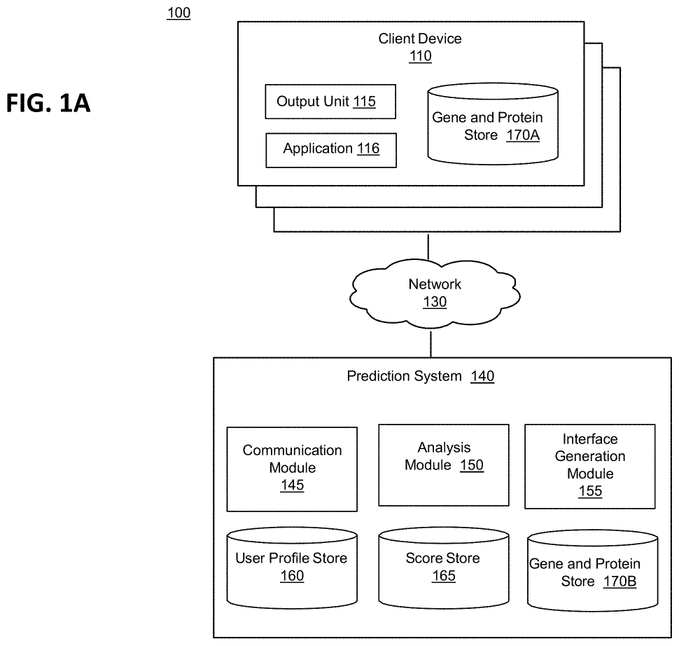

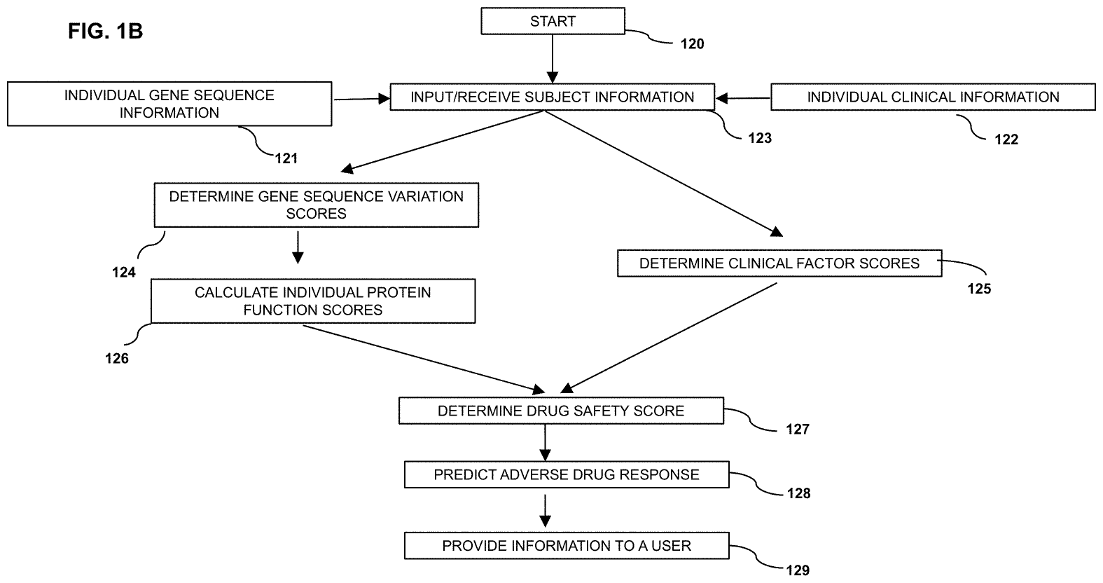

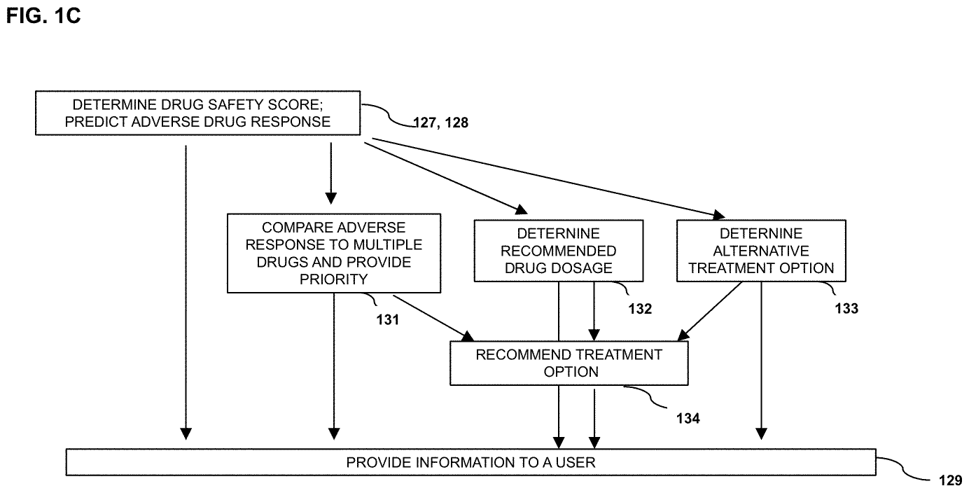

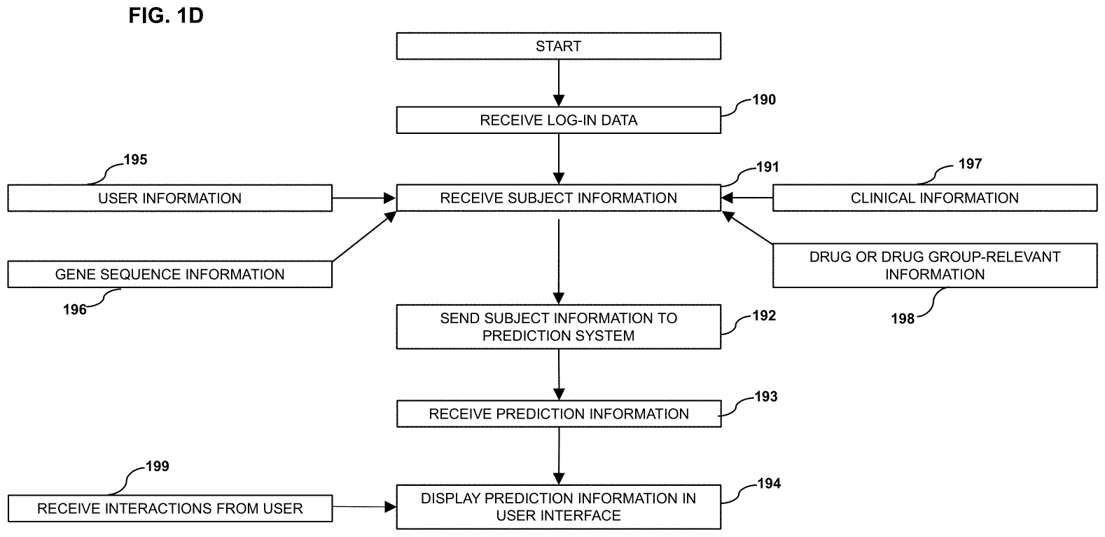

The method of ADR prediction provided herein can be implemented on a computer system. FIG. 1A provides an exemplary system environment 100 for predicting and providing information related to ADR, in accordance with an embodiment. The system environment 100 may include one or more client devices 110 and a prediction system 140 connected to each other over a network 130.

The exemplary system of FIG. 1A is provided by way of illustration not limitation. In other embodiments, different and/or additional entities can be included in the system environment. For example, in some embodiments, the prediction system may not communicate with a user or a client device by a network, and instead can receive client information or transmit prediction information to the user using computer-readable medium, such as ROM (read only memory); a RAM (random access memory); a magnetic disc storage medium; an optical storage medium; a flash memory device; and other electric, optical or acoustic storage medium. In some embodiments, the prediction system can receive client information or transmit prediction information as a hard copy, for example, on a paper.

As one high level example of how the system operates, a physician may have a patient with a particular disease, and the physician is determining which treatment to apply and which drug to prescribe. The patient provides a blood or other bodily sample to a sequencing facility or laboratory (at the physician's office or hospital, at an independent sequencing facility, at a sequencing facility directly associated with the prediction system, etc.), and gene sequence information is determined by the sequencing facility. This data may also have been determined a while ago, and is currently stored by the physician's computer systems, by the patient, or by another provider. The gene sequence variation information is provided to the prediction system 140. In some embodiments, the prediction system 140 receives certain other data, such as clinical information (e.g., electronic medical record (EMR) data) from the physician or hospital about the patient or from the patient herself, clinical guideline data from third party data sources, drug information from third party data sources, etc. The prediction system then performs an analysis on the gene sequence variation information and/or clinical information, including computing one or more scores that provide information about one or more proteins known to relate to the pharmacokinetics or pharmacodynamics of a drug. The system also computes scores and makes a prediction about ADRs of the subject. In some embodiments, the system further computes a drug safety score (DSS) providing information about a drug commonly used for treating the disease and how action of the drug may be affected by the determined functional information about the one or more proteins. In some embodiments, the system combines this information along with patient clinical or EMR data and drug and clinical guideline information from third party sources to determine a recommendation for treatment or to provide basic feedback about the drug that is personalized to that particular patient's protein activity profile. The prediction system can provide any portion of or all of this data (protein function score, drug safety score, predictions made, treatment recommendations, etc.) to the physician, which can be displayed on a computer in a user interface to the physician that the physician can use to determine the best course of treatment for the patient.

As another high level example of how the system operates, the system can be used in designing a clinical trial protocol or interpreting data from clinical study. Clinical trials test potential treatments in human volunteers to see whether they should be approved for wider use in the general population. Typically, clinical studies are conducted without considering variations in the genes associated with adsorption, metabolism, action and excretion of a drug in a population. As a result, a subpopulation with a high pharmacogenetic risk may be under-represented in small-scale clinical studies, and not all the side effects of a drug are discovered through clinical studies. In fact, there have been a number of drugs which were once released in the market but later withdrawn because of side effects which had not been found through the clinical studies. Previously, high-risk subpopulations have generally not been identified when conducting or analyzing clinical studies.

Genetic analysis enables prediction of response to drugs or chemicals. For example, genetic differences (e.g., genetic polymorphism of enzymes involved in drug metabolism) have been associated with efficacy or side effects of a number of drugs. The efficacy or side effects of a drug may be different among individuals because drug metabolism can be slower or faster depending on the particular genetic variations of the individuals.

Researchers have carried out studies in this regard to identify drug responses associated with genetic variations, in addition to identifying the severity of diseases to be treated, drug-drug interactions, and also the age, nutritional condition, and liver/kidney function of a patient, along with environmental factors for a patient, such as climate or food. For example, researchers have examined efficacy of certain drugs in patients with chronic conditions by evaluating the effect of polymorphisms in select candidate genes on the response of patients to the drugs. In addition, pharmacogenetics or pharmacogenomics based studies on the interrelationship between genomic information such as a single-nucleotide polymorphism (SNP) as markers and drug response/side effects, etc. have been done.

However, it has been difficult to find such genetic markers for each drug that allows prediction of response to each drug. Responses to drugs usually result from a complex interplay between genetic variations in individuals' genome sequences, the drug, and various other factors which are difficult to control or be identified. A drug associated with a larger variability of related genes is more likely to cause diverse drug responses. Prior work does not provide useful and reliable drug information for various subpopulations beyond methods based on observational studies of a population using markers, such as single-nucleotide polymorphisms.

Moreover, any drug approved by the FDA and sold in the market can be ordered to be withdrawn from the market according to a result of a post-market surveillance (PMS) while being widely used. Such withdrawal of a drug from the market is a medically critical issue. Even a drug approved after the whole process of a strict clinical trial may cause unpredicted side effects in an actual application step with enormous sacrifices of life and economic losses and thus may be withdrawn. Differences in individual responses which cannot be found even with a large-scale clinical trial are regarded as one of the causes for withdrawal of a drug from the market.

The prediction system described herein allows a way to selectively treat patients based on the predicted response to the drug. This enables safe and personalized use of the drug.

Furthermore, the system can be used for the unapproved drugs during their clinical trial. By selecting a population who will get a benefit from the drug and running clinical trial targeting the patient population, drug developers can avoid high-risk patient groups and increase the likelihood to show safety and effectiveness of tested drugs.

For example, subjects with a DSS score indicating a low adverse drug reaction (e.g., below a threshold value) may be assigned to a low-risk subpopulation for a drug, and subjects with a DSS score indicating a high adverse drug reaction (e.g., above a threshold value) may be assigned to a high-risk subpopulation for the drug. A clinical trial may target subjects that are assigned to low-risk subpopulations, and subsequently, physicians may prescribe the drug to patients belonging to the low-risk group, adjust dosages or the drug depending on whether or not a subject belongs to a high-risk group or a low-risk group, or entirely exclude the drug from a subject who belongs to a high-risk group. Thus, the prediction system provides a way to identify certain segments of the population that are likely to have or not have adverse reactions to the drug in advance, such that the efficacy and safety of a drug can be thoroughly assessed before the drug is given to subjects.

### 5.3.1. Network

The network 130 facilitates communications between one or more client devices 110, and the prediction system 140. The network 130 includes any combination of local area and/or wide area networks, using both wired and/or wireless communication systems. In one embodiment, the network 130 uses standard communications technologies and/or protocols. For example, the network 130 includes communication links using technologies such as Ethernet, 802.11, worldwide interoperability for microwave access (WiMAX), 3G, 4G, code division multiple access (CDMA), digital subscriber line (DSL), etc. Examples of networking protocols used for communicating via the network 130 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over the network 130 may be represented using any suitable format, such as hypertext markup language (HTML) or extensible markup language (XML). In some embodiments, all or some of the communication links of the network 130 may be encrypted using any suitable technique or techniques.

### 5.3.2. Client Device

The client device 110 is an electronic device such as a personal computer (PC), a desktop computer, a laptop computer, a notebook, a tablet PC executing an operating system, for example, a Microsoft Windows-compatible operating system (OS), Apple OS X, and/or a Linux distribution. In another embodiment, the client device 110 can be any device having computer functionality, such as a personal digital assistant (PDA), mobile telephone, smartphone, etc. The client device 110 transmits and receives data, such as patient information and/or sequence information, via the network 130. The client device 110 can be a local device associated with or managed by the prediction system, or a remote device owned or managed by a third party entity, such as a physician, a hospital, a gene sequencing facility, a laboratory, a research facility, etc. For example, the prediction system may receive data over the network from a client device of a sequencing facility, and may send data over the network to a physician's mobile phone or to a physician or hospital computer system.

In addition, the client device 110 can be a third party computer system that collects and/or stores data about different drugs, such as a medication information library that might be maintained by the Food and Drug Administration (FDA) or various other sites that curate data about medications and their affects, what genes and/or proteins are affected by the medications, how medications affect certain organs of the body, pharmokinetic and pharmacodynamics data about a medication, etc. The prediction system 140 can receive information from these third party libraries of data that can be stored and/or used by the prediction system 140 in making a prediction as to how a detected protein deficiency in a subject might affect that subject's response to a given medication.

Other libraries that can be utilized by the prediction system 140 include libraries storing data including clinical guidelines for physician treatment of patients with particular conditions, in determining diagnosis or prognosis, in prescribing certain drugs, etc. This information can be used by the prediction system 140 to provide or recommend a course of action to a physician or caregiver about how to respond to data determined by the prediction system 140 about activity of a protein for a particular patient. For example, based on a functional deficiency predicted for a given patient, the prediction system can predict that the patient will have an adverse effect if prescribed a particular drug, so the system can recommend to the physician not to prescribe that drug or to limit the prescription to a particular dosage, and can also propose alternative drugs/dosages.

As depicted in FIG. 1A, the client device 110 can include an output unit 115, an application 116, and a gene and protein store 170A. In one embodiment, the output unit 115 is embodied as a display on the client device 110. In various embodiments, the client device 110 allows a user to provide input that can be transmitted through the network 130 to the prediction system 140. For example, the client device 110 is capable of transmitting user input from a user, such as a physician, a patient, a pharmacist, or any other user having relevant patient information, through the network 130.

In some embodiments, a client device 110 executes an application 116 allowing a user of the client device 110 to interact with the prediction system 140. Such an application can be created by the prediction system 140 and installed on the client device 110. The output unit 115 can display data received from the prediction system 140 as a representation in a user interface of the application on the client device 110 that is configured to present the data in an easy to interpret format for the user controlling the client device 110. In various embodiments, a user of the client device 110 creates login credentials (e.g., user identifier and password) using the application installed on the client device 110.

The output unit 115 can provide information received from the prediction system 140 to a user of the client device 110. In some embodiments, the client device 110 combines information received from the prediction system 140 with information stored in the gene and protein store 170A to generate information to be provided to the user. As one example, the gene and protein store 170A can include information related to drugs, diseases, or biological functions related to the protein analyzed by the prediction system 140. Information stored in the gene and protein store 170A can be also related to diseases associated with functional changes of analyzed proteins, or susceptibility to such diseases, drug responses, or prognosis to various diseases, etc. In some embodiments, the drug and gene store can be solely embodied in the prediction system 140 as gene and protein store 170B and is not included in the client device 110.

### 5.3.3. Prediction System

As shown in FIG. 1A, the prediction system 140 includes various modules such as a communication module 145, an analysis module 150, and an interface generation module 155. Additionally, the prediction system 140 includes a user profile store 160, a score store 165, and a gene and protein store 170B. In other embodiments, the prediction system 140 may include additional, fewer, or different modules for various applications.

The prediction system 140 receives genetic and/or clinical information of a subject and predicts relevant phenotype based on the received information. Subsequently, the prediction information can be provided to a client device 110 to be presented on the output unit 115. As an example, the prediction system 140 calculates a drug safety score and sends the information to the client device. The drug safety score can be further analyzed in the analysis module 150 before being sent to the user, for example, using information in gene and protein store 170B. For example, the analysis module can use the drug safety score to generate information related to a response of the patient to a drug, choose a better drug or treatment option for the patient, or determine a prognosis of the patient to a disease with or without treatment. The information generated in the prediction system can be assembled and transmitted to the client device 110 for presentation to a user of the client device 110.

The analysis module 150 can further conduct retrospective analysis using genomic, clinical, and phenotypic data from database, such as the UK Biobank (https://www.ukbiobank.ac.uk/). The retrospective analysis can involve identification of correlations between gene sequence and clinical information and adverse drug responses. The analysis module 150 can perform machine learning to identify weightings assigned to genetic variations or clinical factors for determination of drug responses. The weightings can be correlated with importance of each genetic or clinical factor in prediction of drug responses. For example, a larger weight is assigned to a genetic or clinical factor having a high level of correlation with an adverse drug response, and a smaller weight is assigned to a genetic or clinical factor having a low level of correlation with an adverse drug response. In some embodiments, the analysis module 150 receives weightings determined by machine learning from entities external from the prediction system 140.

The communication module 145 controls communication between the prediction system 140 and entities external to the prediction system 140, such as communication over the network 130 with the client device 110, communication with third party systems to retrieve drug information, clinical information, health guidelines for treatment of patients, drug responses of patients, correlation between genetic and clinical information and drug response, weightings assigned to each genetic or clinical factor for prediction of drug response, etc. In one embodiment, the communications module 145 is a wired or wireless interface that manages data transmitted to and from the prediction system 140.

In one embodiment, the communication module 145 receives user login credentials from the client device 110 and verifies the login credentials. To verify the login credentials, the communication module 145 can query the user profile store 160 that stores multiple user profiles. In various embodiments, each user profile is associated with a physician and therefore, user profile information can include patient information for the patients of the physician.

In one embodiment, the communication module 145 receives patient information from the client device 110. In another embodiment, the communication module 145 receives patient information from a third party (e.g., a lab) that performs or maintains laboratory tests (e.g., gene sequencing or other lab test results) for identifying patient information. For example, the communications may receive gene sequence information via the communications module 145. This data may be received from a sequencing facility that has sequenced the patient's data based on a blood or other sample that the patient provided to the sequencing lab for analysis and determination of the genetic sequence and sequence variations of the patient. The sequencing facility may be associated with the patient's physician or hospital, or may be an independent facility to which the patient provided a sample to receive the sequencing data. As another example, the prediction system 140 can be operated in a facility that includes a sequencing laboratory that receives a sample from a patient, and determines sequencing information based on the sample in a laboratory of the prediction system facility from which the prediction system receives the gene sequence variation information.

Patient information or subject information can refer to gene sequence information (e.g., nucleotide sequences) from a sample obtained from the patient. In one embodiment, the communication module 145 receives DNA sequences of the patient that can be used to identify gene sequence variation information. In some embodiments, the analysis of the patient's DNA sequences is conducted by a third party (e.g., a lab) such that the communication module 145 receives the gene sequence variation information from the third party. Patient or subject information can further include clinical information such as patient medical history, biographical information about the patient (age, gender, height, weight, race, cultural ethnicity, non-smoker, etc.), vital sign data and history (blood pressure, heart rate, temperature, oxygen level, etc.). data about past medical treatments and conditions of the patient, current symptoms, prior diagnoses or prognoses, medical images taken of the patient, drug or treatments currently or previously used, adherence by the patient to treatments, etc. The communication module 145 provides the patient information to the analysis module 150 to be used to predict various phenotypes of the patient.

Generally, the gene sequence variation information is information related to substitution, addition, or deletion of a nucleotide within an exon of a gene from the patient. In some embodiments, the gene sequence variation information is information related to substitution, addition, or deletion of a nucleotide within an intron or a regulatory sequence of a gene from the patient. In some embodiments, the substitution, addition, or deletion of the nucleotide results from breakage, deletion, duplication, inversion or translocation of a patient's chromosome or a portion of a chromosome. The genome sequence information of individuals used in the present invention may be determined by using a well-known sequencing method. Further, commercially available services such as those provided by Complete Genomics, BGI (Beijing Genome Institute), Knome, Macrogen, DNALink, etc. which provide commercialized services may be used, although not being limited thereto. Gene sequence variation information present in the genome sequence information of patients may be extracted by using various methods and may be acquired through sequence comparison analysis by using an algorithm such as ANNOVAR (Wang et al., Nucleic Acids Research, 2010; 38(16): e164), SVA (SequenceVariantAnalyzer) (Ge et al., Bioinformatics, 2011; 27(14): 1998-2000), BreakDancer (Chen et al., Nat Methods, 2009 Sep; 6(9): 677-81), etc., which compares a sequence to a reference group.

The gene and protein store 170B stores the types of information described above that can be stored by the store 170A on the client device, including for example gene sequence variation information received and any other patient information received. It can also store data received from third party systems or libraries, such as clinical guideline or drug data.

The analysis module 150 receives data from the communication module 145 and accesses information in the gene and protein store 170B. It performs the analysis on this data to determine protein function score. The analysis module 150 calculates one or more scores associated with the gene sequence variation information received, including gene sequence variation score, protein function drug score, etc. The details behind computation of these scores is provided below.

The analysis module 150 can further determine clinical factor scores based on individual clinical information from the communication module 145. Clinical factor scores are numerical representation of various clinical factors such as patient medical history, biographical information about the patient (age, gender, height, weight, BMI, race, ethnicity, smoking history, alcohol consumption, etc.), vital sign data and history (blood pressure, heart rate, temperature, oxygen level, etc.), lab data (hemoglobin level, international normalized ratio (INR), serum albumin, AST/ALT ratio, etc.) data about past medical treatments and conditions of the patient, current symptoms, prior diagnoses or prognoses, medical images taken of the patient, drug or treatments currently or previously used, concurrent medication, adherence by the patient to treatments, etc.

The analysis module 150 then makes a prediction about drug activity and response for the patient based on the protein function scores and/or clinical factor scores.

The score store 165 stores the various scores computed by the analysis module 150, including gene variation score, protein function score, clinical factor score, drug safety score, etc. The store 165 can also store any prediction made by the module 150 based on the scores.

The interface generation module 155 receives data from the analysis module 150 and configures it for providing a representation of the data in an interface. This data may be provided in an interface on a local computer associated with the prediction system 140, may be sent over the network to be provided in an interface on a remote computer within an installed application that interacts with the prediction system, or both. In some embodiments, the communication module 145 transmits an interactive user interface (UI) (or data for an interactive UI) generated by the interface generation module 155 through the network 130 to the client device 110. Here, the interactive UI includes phenotype information generated by the analysis module 150, as is described in further detail below. In some embodiments, the communication module 145 transmits, to the client device 110, instructions generated by the interface generation module 155 along with the phenotype information generated by the analysis module 150. In this scenario, the client device 110 generates the interactive UI that includes the phenotype information based on the instructions. The user interface can include images and links that a user can click through to access different levels of information illustrating the potential effects of the drug on the patient and providing more details about the ADR that might occur. The user can access click through the UI (including various menus or links) to view pharmacodynamic and pharmacokinetic information about the drug, can view images of organs and how each is affected by the drug, can view specific data about the patient to better understand how the particular patient might be affected by the drug, among other types of data.

There are various methods that are performed by systems of the present invention. FIG. 1B provides an exemplary flowchart illustrating a method of calculating a protein function score and a clinical factor score and using the scores for the treatment of a patient according to an exemplary embodiment of the present invention. In this example, subject information is received 123 by the system, where the subject information includes individual gene sequence information 121, and clinical information 122 of the subject. The individual gene sequence information and clinical information can be input or received from a laboratory or sequencing facility or from stored data about the patient. The system calculates 124 a set of gene sequence variation scores using the individual gene sequence information. The system then calculates an individual protein function score 126 using the set of gene sequence variation scores. The system also determines clinical factor scores 125 using the individual clinical information. The system uses the gene sequence variation scores and clinical factor scores to determine a drug safety score 127. The drug safety score can be used to predict adverse drug response 128 of the subject. The information related to the adverse drug reaction can be provided or sent 129 for display on a client device for treatment of the subject.

The drug safety score 127 and predicted adverse drug response 128 can be further processed before being sent to a user as illustrated in FIG. 1C. For example, the information can be used to compare 131 adverse response of multiple drugs and sort drugs by ranking or determining the order of priority among drugs according to the rankings. For example, drugs can be ordered by their risk to the patient or by their likelihood of being effective for the patient, or by considering both. The drug safety score 127 and predicted adverse drug response 128 can be also used to determine optimal drug dosages for the subject or to determine alternative treatment or drug options. The information can be provided to a user, or can be used to recommend 134 a treatment option for the subject.

One of more of the scores calculated or other information determined (recommendation for treatment, adverse drug reaction, etc.) can then be provided or sent for display on a client device. This data can be prepared or organized by the system for display on a user interface, such as the interactive UI described above.

FIG. 1D is a flowchart illustrating steps performed by an application running on a third party (e.g., a physician or medical staff, a patient) device that is interacting with the prediction system 140. These steps can occur in different orders than what is presented here, and can include fewer or additional steps than what is shown. In some embodiments, the process begins with the application receiving 190 login data from a user, such as a physician or other medical staff, to login to the application (though in some cases login information is not required). The application receives 195 from the user information about the patient about whom an analysis is going to be conducted, including biographical information or unique patient identifier information identifying the patient. In some cases, the application can also retrieve 197 EMR data stored in the physician's database about the patient and their medical history. The application also receives 198, in some embodiments, information about one or more drugs the physician is considering prescribing and the current condition or disease for which the patient is being treated. The application further receives gene sequence variation data 196 and clinical information 197 of a patient. In some embodiments, the physician can get a sample or coordinate the getting of a sample from a patient and provide it to a laboratory at which the gene sequence information or clinical information can be determined, and the physician can receive this data from the laboratory.

The application then sends 192 the gene sequence variation information to the system for analysis. The application can further send 192 various other patient information (e.g., EMR information, information about the drug(s) being considered for treatment, etc.) to the system. The application receives 193 prediction information from the system, which can include one or more scores computed, prediction information, phenotype information, and/or treatment recommendations from the system. The application then displays 194 the prediction information in a user interface (e.g., an interactive UI) for the user to view and interact with. In some cases, the application receives 199 various interactions from the user with the prediction information across one or more UI displays. The application can allow the user to drill down to get additional details about the information, can perform calculations based on the information, and can output recommendations to the user for treatment of the patient.

### 5.4. GENE SEQUENCE VARIATION INFORMATION

The gene sequence variation information used in embodiments of the present invention refers to information related to an individual gene sequence variation or polymorphism. In some embodiments, the gene sequence variation or polymorphism occurs particularly in an exon region of a gene encoding proteins, but is not limited thereto. In some embodiments, the gene sequence variation or polymorphism occurs particularly in an intron region or a regulatory sequence of a gene.

A polymorphism of a sequence refers to individual differences in their genomic sequences. In particular, single nucleotide polymorphisms (SNPs) are commonly found. The single nucleotide polymorphism refers to individual differences in one base of a sequence consisting of A, T, C, and G bases. The sequence polymorphism including the SNP can be expressed as a SNV (Single Nucleotide Variation), STRP (short tandem repeat polymorphism), or a poly-allelic variation including VNTR (various number of tandem repeat) and CNV (Copy number variation).

Sequence variation or polymorphism information can be associated with a protein involved in various phenotypes, such as biological activity, metabolism, diseases or pharmacodynamics or pharmacokinetics of a predetermined drug or drug group. The sequence variation information can be variation information found in an exon of a gene involved in the various phenotypes. For example, the gene can encode a target protein relevant to a drug, an enzyme protein involved in biological activity or metabolism, a transporter protein, and a carrier protein, but is not limited thereto. The sequence variation information can be variation information found in an intron or a regulatory sequence of a gene.

The individual genome sequence information used herein may be determined by using a well-known sequencing method. Further, commercially available services such as Complete Genomics, BGI (Beijing Genome Institute), Knome, Macrogen, and DNALink can be used, but the present invention is not limited thereto.

Gene sequence variation information present in an individual genome sequence can be extracted by using various methods. For example, the information can be acquired through sequence comparison analysis by using a program, such as ANNOVAR (Wang et al., Nucleic Acids Research, 2010; 38(16): e164), SVA(Sequence Variant Analyzer) (Ge et al., Bioinformatics. 2011; 27(14): 1998-2000), BreakDancer (Chen et al., Nat Methods. 2009 Sep; 6(9):677-81), and the like, which compare a sequence to a reference group (e.g., the genome sequence of HG19).

The gene sequence variation information may be received/acquired through a computer system. In this aspect, the method of the present invention can further include receiving the gene sequence variation information through a computer system. The computer system can include or access one or more databases including information about the gene involved in various phenotypes, such as biological activity, metabolism, diseases or pharmacodynamics or pharmacokinetics of a predetermined drug or drug group such as a gene encoding a target protein involved in metabolism, transport, or other processes of the drug or the drug group. These databases may include a public or non-public database or a knowledge base, which provides information about gene/protein/drug-protein interaction, and the like, including DrugBank (http://www.drugbank.ca/), KEGG Drug (http://www.genomejp/kegg/drug/), and PharmGKB (http://www.pharmgkb.org/), but are not limited thereto.

### 5.5. GENE SEQUENCE VARIATION SCORE

The gene sequence variation score can be calculated by using various methods, including some methods known in the art. For example, the gene sequence variation score can be calculated from the gene sequence variation information by using one or more of the algorithms selected from SIFT (Sorting Intolerant From Tolerant, Pauline C et al., Genome Res. 2001 May; 11(5): 863-874; Pauline C et al., Genome Res. 2002 March; 12(3): 436-446; Jing Hulet al., Genome Biol. 2012; 13(2): R9), PolyPhen, PolyPhen-2 (Polymorphism Phenotyping, Ramensky V et al., Nucleic Acids Res. 2002 September 1; 30(17): 3894-3900; Adzhubei IA et al., Nat Methods 7(4):248-249 (2010)), MAPP (Eric A. et al., Multivariate Analysis of Protein Polymorphism, Genome Research 2005; 15:978-986), Logre (Log R Pfam E-value, Clifford R.J et al., Bioinformatics 2004; 20:1006-1014), Mutation Assessor (Reva B et al., Genome Biol. 2007; 8:R232, http://mutationassessor.org/), Condel (Gonzalez-Perez A et al., The American Journal of Human Genetics 2011; 88:440-449, http://bg.upf.edu/fannsdb/), GERP (Cooper et al., Genomic Evolutionary Rate Profiling, Genome Res. 2005; 15:901-913, http://mendel.stanford.edu/SidowLab/downloads/gerp/), CADD (Combined Annotation-Dependent Depletion, http://cadd.gs.washington.edu/), MutationTaster, MutationTaster2 (Schwarz et al., MutationTaster2: mutation prediction for the deep-sequencing age. Nature Methods 2014; 11:361-362, http://www.mutationtaster.org/), PROVEAN (Choi et al., PLoS One. 2012; 7(10):e46688), PMut (Ferrer-Costa et al., Proteins 2004; 57(4):811-819, http://mmb.pcb.ub.es/PMut/), CEO (Combinatorial Entropy Optimization, Reva et al., Genome Biol 2007; 8(11):R232), SNPeffect (Reumers et al., Bioinformatics. 2006; 22(17):2183-2185, http://snpeffect.vib.be), fathmm (Shihab et al., Functional Analysis through Hidden Markov Models, Hum Mutat 2013; 34:57-65, http://fathmm biocompute.org.uk/), VAMP-seq (Matreyek et al., Multiplex Assessment of Protein Variant Abundance by Massively Parallel Sequencing, Nature Genet. 2018; 50(6):874-882), optimized prediction framework (Zhou et al., An optimized prediction framework to assess the functional impact of pharmacogenetic variants, The Pharmacogenomics Journal 2019; 19:115-126), and the like, but the present invention is not limited thereto. Each of the aforementioned references in this paragraph are incorporated by reference herein.

The above-described algorithms can be related to how much effect each gene sequence variation has protein function. These algorithms calculate a score based on a protein sequence encoded by a corresponding gene and changes resulting from variations and thereby determines an effect of the variations on a structure and/or function of the corresponding protein.

In an exemplary embodiment, a SIFT (Sorting Intolerant From Tolerant) algorithm is used to calculate an individual gene sequence variation score. In the SIFT algorithm, gene sequence variation information is input in the form of a VCF (Variant Call Format) file, and a degree of damage caused by each gene sequence variation to the corresponding gene is scored. When the SIFT score is closer to 0, the variation corresponding to the score is considered to cause severe damages to the protein, and when the SIFT score is closed to 1, the corresponding variation is considered to cause less damages to the protein.

In case of PolyPhen-2, on the other hand, the higher a calculated score is, it is considered that the more damaged a protein encoded by a corresponding gene is.

In some embodiments, the gene sequence variation score is an ensemble score integrating assessments obtained by multiple algorithms Such an ensemble score includes CADD, DANN, MetaSVM and MetaLR.

In some embodiments, the gene sequence variation score is obtained integrating and optimizing assessments by multiple individual algorithms based on their overall informedness, defined as the probability that a prediction is informed (i.e., not by chance). An example of such optimized prediction framework is described in Zhou et al., An optimized prediction framework to assess the functional impact of pharmacogenetic variants, The Pharmacogenomics Journal 2019; 19:115-126, which is incorporated by reference in its entirety herein.

In case of ADME-optimized algorithm, thresholds for individual algorithms (including ANNOVAR, SIFT, PolyPhen-2, Likelihood ratio tests, MutationAssessor, FAATHMM, FATHMM-MKL, PROVEAN, VEST3, CADD, DANN, MetaSVM, MetaLR, GERP++, SiPhy, PhyloP, and PhastCons) are determined on the basis of the Youden index or informedness function. Variants are classified as deleterious or neutral by each of the k threshold-optimized algorithms Out of all possible constellations, the algorithm combination are selected for the ADME-optimized model, using functional data of ADME (drug absorption, distribution, metabolism and excretion) gene mutations. The overall prediction score of the ADME-optimized model is determined by predicting, with each algorithm, whether a variant is deleterious or neutral based on its ADME-optimized threshold value (1=deleterious and 0=functionally neutral). The prediction score is derived by averaging the assessments of the individual algorithms, where a score of 1 indicates that all algorithms predicted the variant to be deleterious and a score of 0 indicates that all algorithms predicted the variant to be neutral.

In some embodiments, the gene sequence variation score is a score determined by an experimental wet-lab approach that determines a sequence variation and protein function. For example, the score is obtained by Variant Abundance by Massively Parallel Sequencing (VAMP-seq), which measures the effects of thousands of missense variants of a protein on intracellular abundance simultaneously, as described in Matreyek et al., Multiplex Assessment of Protein Variant Abundance by Massively Parallel Sequencing, Nature Genet. 2018; 50(6):874-882).

In the particular method, a mixed population of cells each expressing one protein variant fused to EGFP is created. The variant dictates the abundance of the variant-EGFP fusion protein, resulting in a range of cellular EGFP fluorescence levels. Cells are then sorted into bins based on their level of fluorescence, and high throughput sequencing is used to quantify every variant in each bin. VAMP-seq scores are calculated from the scaled, weighted average of variants across bins, where a low score indicates low abundance and a high score indicates high abundance. A similar method or a variation thereof can be used to determine functional effects of protein variants.

Recently, a study was performed to compare SIFT, Polyphen2, MAPP, Logre, and Mutation Assessor algorithms and the results were reported. (Gonzalez-Perez, A. & López-Bigas, N. Improving the assessment of the outcome of nonsynonymous SNVs with a consensus deleteriousness score, Condel. The American Journal of Human Genetics, 2011; 88(4):440-449.). In the study, the above-described five algorithms were compared in view of known data available from HumVar and HumDiv (Adzhubei,t IAet al., A method and server for predicting damaging missense mutations. Nature methods, 2010; 7(4):248-249). In the study, 97.9% of the gene sequence variations associated with protein damages and 97.3% of the gene sequence variations associated with less protein damages according to HumVar were commonly identified by at least three of the above five algorithms Similarly, 99.7% of the gene sequence variations associated with protein damages and 98.8% of the gene sequence variations associated with less protein damages according to HumDiv were commonly identified by at least three of the above five algorithms. Further, ROCs (Receiver Operating Curves) generated using the five algorithms based on the HumDiv and HumVar data supported consistency of their calculation results, by having AUC (Area Under the Receiver Operating Curve) highly consistent for the various methods (69% to 88.2%). These demonstrated that gene sequence variation scores calculated by the different methods are significantly correlated to each other. Therefore, gene sequence variation scores calculated by any algorithms can be used in various embodiments of the present invention, for example, to calculate a protein score, a drug score, a prescription score, etc.

When a gene sequence variation occurs in an exon region of a gene encoding a protein, the gene sequence variation may directly affect a structure and/or function of the protein. When a gene sequence variation occurs in an intron or a regulatory region of a gene encoding a protein, the gene sequence variation can affect an expression and/or function of the protein. Therefore, the gene sequence variation information may be associated with a degree of damage to a protein function. In this aspect, the method of the present invention calculates an individual protein function score or an individual protein damage score on the basis of the above-described gene sequence variation score in the following step.

### 5.6. PROTEIN FUNCTION SCORE

Protein function scores are calculated based on gene sequence variation scores.

In some embodiments, the protein function score is calculated as a mean of the selected gene sequence variation scores by calculating, for example, but not limited to, a geometric mean, an arithmetic mean, a harmonic mean, an arithmetic geometric mean, an arithmetic harmonic mean, a geometric harmonic mean, Pythagorean means, an interquartile mean, a quadratic mean, a truncated mean, a Winsorized mean, a weighted mean, a weighted geometric mean, a weighted arithmetic mean, a weighted harmonic mean, a mean of a function, a generalized mean, a generalized f-mean, a percentile, a maximum value, a minimum value, a mode, a median, a mid-range, a central tendency, simple multiplication or weighted multiplication, or by a functional operation of the calculated values.

In an exemplary embodiment of the present invention, the protein function score is calculated by the following Equation 1. The following Equation 1 can be modified in various ways, and, thus, the present invention is not limited thereto.

$${F}_{gk} ( {v}_{1} , … , {v}_{{n}_{k}} ) = {( \frac{1}{{n}_{k}} {\sum }_{j = 1}^{{n}_{k}} {v}_{j , k}^{p} )}^{\frac{1}{p}} \qquad Equation 1$$

In Equation 1, F_{gk} is a protein function score of a protein encoded by a gene gₖ, n is the number of sequence variations of the gene g, vᵢ is a gene sequence variation score of an j^{th} gene sequence variation, and p is a real number other than 0. In Equation 1, when a value of the p is 1, the protein function score is an arithmetic mean, if the value of the p is −1, the protein function score is a harmonic mean, and if the value of the p is close to the limit 0, the protein function score is a geometric mean.

In preferred embodiments, the mean is calculated by measuring a geometric mean. Specifically, the protein function score is calculated by the following Equation 2. Equation 2 is

$${F}_{gk ( or g )} ( {v}_{1} , … , {v}_{{n}_{k}} ) = {( {\prod }_{j = 1}^{{n}_{k}} {v}_{j , k}^{{b}_{j , k}} )}^{\frac{1}{{\sum }_{j = 1}^{{n}_{k}} {b}_{j , k}}}$$

In Equation 2, F_{gk} is a protein function score of a protein encoded by a gene gₖ (F_{g} is a protein function score of a protein encoded by a gene g), nₖ is the number of sequence variations of the gene gₖ, v_{j,k} is a gene sequence variation score of an j^{th} gene sequence variation, and b_{j,k} is a weighting assigned to the v_{j,k}. If all weightings b_{j,k} have the same value, the protein function score F_{g} is a geometric mean of the gene sequence variation scores v_{j,k}. In some embodiments, all weightings b_{j.k} have the same value, e.g., 1. In some embodiments, some weightings are 0.

However, it is appreciated that the protein function score can be calculated as any function of the gene sequence variation scores, and this function may be parameterized by the weightings w_{j,k}. For example, the protein function score may also be an arithmetic mean instead of the geometric mean shown in Equation 2.

In some embodiments, multiple gene sequence variations scores are weighed differently. In some embodiments, the weighting assigned to the gene sequence variation score v_{j,k} is determined based on clinical, medical, biological or demographic information of the subject, or a value of the gene sequence variation score v_{j,k}.In some embodiments, the weighting is determined based on the correlation between each genetic variation score or clinical factor score and the protein function determined by other methods, for example, by a computational or experimental method. In some embodiments, the weighting is determined based on pharmacokinetic parameters Kₘ, Vₘₐₓ, and K_{cat/Km} of the drug. In some embodiments, the weighting is determined based on a characteristic of an interaction between a drug and a protein. For example, a protein directly involved in metabolism of the drug is assigned 2 points, whereas a protein involved in transport of the drug or its metabolite is assigned 1 point. In other example, a target protein and a transporter protein are assigned 2 points and 1 point, respectively.

In some embodiments, only the protein directly interacting with a drug is considered. In some embodiments, the predictive ability of the above Equations 1 and 2 can be improved by using information about the protein interacting with a precursor of the corresponding drug or metabolic products of the corresponding drug, the protein significantly interacting with proteins involved in the pharmacodynamics or pharmacokinetics of the corresponding drug, and the protein involved in a signal transduction pathway thereof. That is, by using information about a protein-protein interaction network or pharmacological pathway, it is possible to use information about various proteins relevant thereto. That is, even if a significant variation is not found in the protein directly interacting with the drug so that there is no protein function score calculated with respect to the protein or there is no damage (for example, 1.0 point when a SIFT algorithm is applied), a mean (for example, a geometric mean) of protein function scores of proteins interacting with the protein or involved in the same signal transduction pathway of the protein may be used as a protein function score of the protein so as to be used for calculating a drug safety score.

In some embodiments, the weighting b_{j,k} assigned to the v_{j,k} is determined by machine learning. The weightings b_{j,k}, j=1,2, . . . nₖ for the gene sequence variations may be determined by training data. The training data includes a plurality of data instances for a protein encoded by gene gₖ. Each training data instance for the protein encoded by gene gk may include the gene sequence variation scores v_{j,k} for gene sequence variations found in the sequence for the protein, and an actual protein function score of how the set of gene sequence variations affected the protein function in that instance.

In one embodiment, the set of weightings b_{j,k}, j=1, 2, . . . , nₖ for the protein function score are determined by repeatedly iterating between steps of calculating a loss function based on the training data and updating the set of weightings to reduce the loss function.

Specifically, at the start of the training process, the values of the set of weightings for the protein function score model are initialized. For each instance of one or more subsets of the training data, an estimated protein function score is generated by inputting the gene sequence variation scores for the training instance to the protein function score model. The estimated protein function score corresponds to an output generated by the protein function score model using the current iteration values of the weightings. For example, the gene sequence variation scores for a training data instance may be input into the protein function score model of Equation 2 to generate an estimated protein function score for the quantity F_{gk}. A loss function that indicates a discrepancy between the actual protein function scores and the estimated protein function scores generated for the training data instances is determined. The set of weightings for the protein function score model is updated to reduce the loss function. This process is repeated until the loss function reaches a predetermined criterion, such as a convergence criterion that is triggered when the loss function changes less than a predetermined threshold.

In one instance, the loss function is given by Equation 2.1:

$$( {pf}_{t ′} , {ef}_{t ′} ; {b}_{j , k} , j = 1 , 2 , … , {n}_{k} ) = {\sum }_{t ′ = 1}^{T ′} ( {‖ {pf}_{t ′} - {ef}_{t } ‖}_{2}^{2} )$$

where pƒₜ, is the actual protein function score for training data instance t′, and eƒₜ, is a corresponding estimated protein function score generated using the gene sequence variation scores for training data instance t′ in the training data set with T′ total instances.

However, it should be appreciated that Equation 2.1 are two possible examples of loss functions, and in practice, any type of loss function that measures the discrepancy between the actual protein function scores and the estimated protein function scores can be used. For example, the loss function may also be a L1 norm, a L∞norm, a sigmoid function, and the like.

Moreover, various minimization or maximization algorithms can be used to repeatedly update the set of parameters b of the prediction model, through gradient-based numerical optimization algorithms, such as batch gradient algorithms, stochastic gradient algorithms, and the like.

### 5.7. CLINICAL FACTOR SCORE

Clinical factor scores are numerical representation of various clinical factors, such as patient medical history, biographical information about the patient (age, gender, height, weight, body mass index (BMI, kg/m²), race, ethnicity, smoking history, alcohol consumption, diet, etc.), vital sign data and history (blood pressure, heart rate, temperature, oxygen level, etc.), lab data (hemoglobin level, international normalized ratio (INR), serum albumin, AST/ALT ratio, etc.) data about past medical treatments and conditions of the patient (detailed transfusion history (PRBCs) including pre/post transfusion HgB/HCT, etc.), current symptoms, prior diagnoses or prognoses, medical images taken of the patient, drug or treatments currently or previously used, concurrent medication, adherence by the patient to treatments, etc.

In some embodiments, medical conditions for consideration are one or more factors selected from the group consisting of hospitalizations, surgeries, emergency room visits, altered gastrointestinal flora, biliary obstruction, cachectic state, collagen disease, diarrhea, hypermetabolic states (fever, hyperthyroidism), hypoalbuminemia, infectious disease, initial hypoprothrombinemia, low dietary vitamin K intake malabsorption states, malignancy, menstruation and menstrual disorders, postoperative status, radiation therapy, renal impairment, scurvy, diabetes, dyslipidemia, edema, gastrointestinal states that impair absorption, hypothyroidism, increased intake or absorption of vitamin K, and visceral carcinoma. In some embodiments, the clinical factors include underlying diseases or medical history related to diabetes, hypertension, dyslipidemia, kidney disease, liver disease, cancers, endocrine disorder, allergies, cardiovascular disease, postoperative status, vaccination status, and autoimmune diseases.

In some embodiments, relevant clinical factors include other treatment or medications concurrently or previously used that could interact with the drug to be administered. For example, clinical factors include prior or concurrent use of any one or more of the drugs selected from amiodarone, abciximab, acetaminophen, alcohol (acute and chronic), allopurinol, alprazolam, aminoglutethimide, amiodarone, amitriptyline, amlodipine, amobarbital, anabolic steroids and sildenafil, apixaban, aripiprazole, aspirin/nonsteroidal anti-inflammatories, atorvastatin, azathioprine, azole antifungals, barbiturate class, benzodiazepines, buspirone, butobarbital, butalbital, carbamazepine, cefoperazone, cefotetan, cefoxitin, ceftriaxone, celecoxib, chemotherapeutic agents, chenodiol, chloral hydrate chloramphenicol, chlorpropamide, chlorthalidone, cholestyramine, cimetidine, ciprofloxacin, clarithromycin, clofibrate, clomipramine, clopidogrel, clozapine, codeine, colestipol, corticotropin, cortisone, coumadin, cyclophosphamide. cyclosporine, danazol, desipramine, dextran, dextromethorphan, dextrothyroxine, diazepam, diazoxide, diclofenac, dicloxacillin, diflunisal, diltiazem, disulfiram, doxycycline, duloxetine, erythromycin, ethacrynic acid, ethchlorvynol, ethinylestradiol, felodipine, fenofibrate, fenoprofen, fluconazole, fluorouracil, Fluvastatin, gemfibrozil, glipizide, glucagon, glutethimide, griseofulvin, haloperidol, halothane, heparin, ibuprofen, ifosfamide, imatinib, imipramine, indinavir, indomethacin, influenza virus vaccine, Irbesartan, itraconazole, ketoprofen, ketorolac, lansoprazole, leflunomide, levamisole, levothyroxine, liothyronine, losartan, lovastatin, mefenamic, meprobamate, methimazole, methyldopa, methylphenidate, methylsalicylate, metoprolol, metronidazole, mexiletine, miconazole, midazolam, moricizine, nafcillin, dicloxacillin, nalidixic acid, naproxen, neomycin, nifedipine, nisoldipine, nitrendipine, norfloxacin, ofloxacin, olsalazine, omeprazole, ondansetron, oxaprozin, oxymetholone, paraldehyde, paroxetine, penicillin G, pentobarbital, pentoxifylline, phenobarbital, phenprocoumon, phenylbutazone, phenytoin, phytonadione, piperacillin, piroxicam, prednisone, primidone, propafenone, propoxyphene, propranolol, propylthiouracil, psyllium, quinidine, quinine, ranitidine, rifampin, risperidone, ritonavir, rivaroxaban, saquinavir, secobarbital, sertraline, simvastatin, sirolimus, spironolactone, stanozolol, streptokinase, sucralfate, sulfamethizole, sulfamethoxazole, sulfinpyrazone, sulfisoxazole, sulindac, tacrine, tacrolimus, tamoxifen, tetracycline, theophylline, thioridazine, thyroid hormone, ticarcillin, ticlopidine, timolol, tolbutamide, torasemide, tramadol, trazodone, triazolam, trimethoprim-sulfamethoxazole, urokinase, valproate, venlafaxine, verapamil, vitamin c, vitamin e, warfarin

In some embodiments, relevant clinical factors include any one or more of the lab test results selected from the group consisting of: measurements of CBC (Haemoglobin RBC & hematocrit (Hct), WBC (white blood cells, leukocytes), Platelets, WBC differentiation . . . etc), PT/aPTT, Renal function (Creatinine, BUN), urinalysis, metabolic panel, lipid panel (Total cholesterol, Triglycerides, HDL-cholesterol, LDL-cholesterol), liver function test, hormone levels (FSH, LH, Estradiol Progesterone Prolactin Testosterone), Diabetes (Glucose, Glycohemoglobin, Insulin C-peptide), Cultures (blood, urine, CSF . . . etc), Allergy (RAST, Total IgE), Anemia (Iron (Fe) Fe & TIBC (total iron binding capacity), Ferritin, Transferrin, Vitamin B12 & folic acid), Inflammation (Blood sedimentation, erythrocyte sedimentation rate (ESR) ,C-reactive protein (CRP), Fibrinogen), Ions (Sodium (Na), Bicarbonate, Potassium (K), and Chloride (Cl).

In some embodiments, relevant clinical factors include any one or more of the imaging results selected from the group consisting of Radiograph, CT, MR, PET, SPECT, Ultrasound, EKG, and DEXA.

In some embodiments, relevant clinical factors include any one or more of the family medical history selected from the group consisting of: genetic disorder, cancer, diabetes, hypertension, dyslipidemia, cardiovascular, and other medical history of family members.

In some embodiments, relevant clinical factors include any one or more of the environmental factors such as smoking, alcohol intake, diet (food-drug interaction), occupation (exposure to certain chemicals), and travel history.

Specific clinical factors for use in the prediction method vary depending of the drug and the condition for treatment. For example, in case of warfarin, clinical factors for consideration include use of amiodarone, aspirin/nonsteroidal anti-inflammatories, trimethoprim-sulfamethoxazole, ciprofloxacin, erythromycin, metronidazole, azole antifungals, prednisone, leflunomide, gemfibrozil, fenofibrate, chemotherapeutic agents, doxycycline, rifampin, carbamazepine, cholestyramine, colestipol, psyllium, barbiturate class, nafcillin and/or dicloxacillin. In case of warfarin, clinical factors for consideration can further include lab test results, such as international normalized ratio (INR), PT/aPTT ratio, Renal function (Creatinine, BUN), liver function test (LFT), CBC, and various hormone levels (TSH, FSH, LH, etc.).

In some embodiments, clinical factors, such as weight, height, body mass index (kg/m²), age, biological sex, ethnicity, and amiodarone use (drug-drug-interaction), are used for the prediction method provided herein.

Clinical factor scores can be determined as a unit number or as a number indicating a relevant category (e.g., smoking can be presented by units of pack-per-year or pack-per-day or categorically such as ex-smoker, occasional smoker, regular smoker, etc.). Some clinical factors can be combined to provide a single clinical factor score (e.g., height and weight can be included separately or converted into body mass index (BMI) or body surface area (BSA) depending on the drug or disease being studied. Some clinical factors can be centered and/or scaled (e.g., weight, height, etc.). In some embodiments, a single clinical factor score is assigned for each clinical factor.

In some embodiments, clinical factor scores are a number within a specified range, for example, between 0 and 1, 0 and 10, or 0 and 100. In some embodiments, all the clinical factor scores used in the determination of adverse drug response are within the specified range. In some embodiments, only some of the clinical factor scores are within the specified range.

### 5.8. DRUG SAFETY SCORE

Drug safety scores are calculated based on protein function scores and/or clinical factor scores described herein. Drug safety scores can be calculated by a prediction model using Equation 3:

DSS=ƒ_{w}(Sᵢ, i=1, 2, . . . , H)

where ƒ is any function parameterized by a set of parameters w, DSS is the drug safety score, Si is a factor (i) relevant to the drug response (e.g., a protein function score, a clinical factor score). In one embodiment, the drug safety scores can be calculated by Equation 4:

$$ln ( \frac{D S S}{1 - D S S} ) = {\sum }_{i = 1}^{H} {w}_{i} {S}_{i}$$

wherein wᵢ is a weighting assigned to the score, and H is the total number of factors considered and included in determination of the drug safety score. Thus, the set of parameters w in Equation 4 are characterized by wᵢ, i=1, 2, . . . , H. Equation 4 can be written as Equation 5:

$$DSS = \frac{1}{1 + {e}^{- ( {\sum }_{i = 1}^{H} {w}_{i} {S}_{i} )}}$$

A drug safety score for a drug can be also calculated by considering both protein function scores and clinical factor score as the factors Sᵢ relevant to the drug response. In some embodiments, each of the protein functions scores and clinical factor scores is relevant to pharmacokinetics or pharmacodynamics of the drug. In these embodiments, the drug safety score can be calculated by using Equation 6, wherein the Equation 6 is

DSS=ƒ_{w}(F_{gk}, k=1, 2, . . . , m; S_{ci}, i=1, 2, . . . , p)

where F_{gk} is the protein function score of the protein encoded by gene gₖ, m is the total number of proteins included for the model, cᵢ is the clinical factor included for the calculation of DSS, and p is the total number of clinical factors included for the calculation of DSS. In one embodiment, the drug safety score can be calculated by using Equation 7, wherein the Equation 7 is:

$$ln ( \frac{D S S}{1 - D S S} ) = {B}_{0} + {W}_{g 1} {F}_{g 1} + {W}_{g 2} {F}_{g 2} + ⋯ {W}_{g m} {F}_{g m} + {W}_{c 1} {S}_{c 1} + {W}_{c 2} {S}_{c 2} + ⋯ {W}_{c p} {S}_{c p}$$

wherein B₀ is the intercept, W_{gk} is a weighting assigned to the protein function score F_{gk}, W_{ci} is a weighting assigned to each clinical factor score S_{ci}. Thus, the set of parameters w in Equation 7 are characterized by the weights W_{gk}, k=1 ,2, . . . , m and W_{ci}, i=1 ,2, . . . p, and wherein the factors Sᵢ referred to in Equation 4 are the protein function scores F_{gk}and the clinical factor scores S_{ci}. Equation 7 can also be written as Equation 7.1:

$$D S S = \frac{1}{1 + {e}^{- ( {B}_{0} + {W}_{g 1} {F}_{g 1} + {W}_{g 2} {F}_{g 2} + ⋯ {W}_{g m} {F}_{g m} + {W}_{c 1} {S}_{c1} + {W}_{c2} {S}_{c2} + ⋯ {W}_{c p} {S}_{c p} )}}$$

While the structure of the parameterized function ƒ_{w}(·) is illustrated in Equations 4 through 7.1 as a logistic regression model, in which the output of the model is generated by a sigmoid of a weighted linear combination of the factors Sᵢ, it should be appreciated that this is one example of the parameterized function. In other embodiments, the parameterized function ƒ_{w}(·) can be structured as any prediction model, including various types of machine-learned models. The machine-learned models may include decision-tree based models, such as gradient-boosted trees, random forests, and the like, neural-network based models such as artificial neural networks (ANN), convolutional neural networks (CNN), deep neural networks (DNN), and the like, additive models such as linear regression models, logistic regression models, step-wise logistics regression models, support vector machine (SVM) models, and the like.

In some embodiments, the total number of proteins (m) included for the calculation of the drug safety score is at least 1, at least 2, at least 3, at least 4, at least 5, at least 6, at least 7, at least 8, at least 9, at least 10, at least 15, at least 20, at least 30, at least 40, at least 50, at least 60, at least 70, at least 80, at least 90 or at least 100. In some embodiments, the total number of proteins (m) included for the calculation of the drug safety score is between 1 and 5, between 1 and 10, between 1 and 20, between 1 and 30, between 1 and 40, between 1 and 50, or between 1 and 100. In some embodiments, the total number of proteins (m) included for the calculation of the drug safety score is between 10 and 50, between 25 and 75, or between 50 and 100.

In some embodiments, the total number of clinical factors (p) included for the calculation of the drug safety score is at least 1, at least 2, at least 3, at least 4, at least 5, at least 6, at least 7, at least 8, at least 9, at least 10, at least 15, at least 20, at least 30, at least 40, at least 50, at least 60, at least 70, at least 80, at least 90 or at least 100. In some embodiments, the total number of clinical factors (p) included for the calculation of the drug safety score is between 1 and 5, between 1 and 10, between 1 and 20, between 1 and 30, between 1 and 40, between 1 and 50, or between 1 and 100. In some embodiments, the total number of clinical factors (p) included for the calculation of the drug safety score is between 10 and 50, between 25 and 75, or between 50 and 100.

The set of parameters for the prediction model can be determined by machine learning using training data containing gene sequence information and clinical information of subjects, and their drug response data. For example, the training data can be obtained from database such as UK Biobank or similar to UK Biobank can be used for the machine learning. Details of how the set of parameters w in the prediction model are trained are provided below.

The training data includes a plurality of data instances t=1, 2, . . . , T, in which each data instance t contains a set of instances of patient data for a corresponding patient and whether the patient had an ADR. Specifically, each data instance t may include the factors Sᵢ relevant to the drug response for the patient, and an actual outcome of whether the patient for the data instance t had an ADR. For example, the factors Sᵢ may be the protein function scores F_{gk} and the clinical factor scores S_{ci} for the patient. In such an example, the protein function scores F_{gk} may be determined from the patient's relevant genome sequence and inputting the variations in the patient's genome to the Equation 2 to generate the protein function scores F_{gk}. The clinical factor scores S_{ci} may be obtained from the patient's clinical data.

In one embodiment, the actual outcome of the data instance t may be encoded as a binary variable indicating whether or not the patient had an ADR. For example, the actual outcome may be 0 if the patient did not suffer an ADR, and 1 if it is determined that the patient did suffer an ADR (e.g., when high DSS predictions correspond to high likelihood of ADR). In another embodiment, the actual outcome of the data instance t may be encoded as a continuous numerical variable indicating a degree to which the patient had suffered ADR. For example, the actual outcome may be a numerical value between 0 and 1, in which 0 indicates no ADR, 1 indicates highest degree of ADR, and values in between denote varying degrees of ADR.

In one embodiment, the set of parameters for the prediction model are determined by repeatedly iterating between calculating a loss function based on the training data and updating the set of parameters to reduce the loss function.

Specifically, at the start of the training process, the values of the set of parameters for the prediction model are initialized. For each instance of one or more subsets of the training data, an estimated output is generated by inputting the factors for the training instance to the prediction model. The estimated output corresponds to an output generated by the prediction model using the current values of the parameters. For example, the protein function scores F_{gk} and the clinical factor scores S_{ci} for the patient may be input into the prediction model of Equation 7.1 to generate an estimated output for the quantity DSS. A loss function that indicates a discrepancy between the actual outcomes for the training data instances and the estimated outputs generated for the training data instances is determined. The set of parameters for the prediction model are updated to reduce the loss function. This process is repeated until the loss function reaches a predetermined criterion, such as a convergence criterion.

In one instance, the loss function is the negative log likelihood function given by Equation 8:

$$( {a}_{t} , {e}_{t} ; w ) = {\sum }_{t = 1}^{T} ( {a}_{t} log {e}_{t} + ( 1 - {a}_{t} ) log ( 1 - {e}_{t} ) )$$

where aₜ is the actual outcome for training data instance t, and eₜ is a corresponding estimated output generated using the factors for training data instance t. As indicated in Equation 8, the loss function may be a combination across the training data instances in a subset of training data t=1, 2, . . . , T. In another instance, the loss function is a L2 norm given by Equation 9:

$$( {a}_{t} , {e}_{t} ; w ) = {\sum }_{t = 1}^{T} ( {‖ {a}_{t} - {e}_{t} ‖}_{2}^{2} )$$

However, it should be appreciated that Equations 8 and 9 are two possible examples of loss functions, and in practice, any type of loss function that measures the discrepancy between the actual outcome and the estimated outputs can be used. For example, the loss function may also be a L1 norm, a L∞ norm, a sigmoid function, and the like.

Moreover, various minimization or maximization algorithms can be used to repeatedly update the set of parameters w of the prediction model, through gradient-based numerical optimization algorithms, such as batch gradient algorithms, stochastic gradient algorithms, and the like.

In one embodiment, training data instances in the loss function are weighted according to the actual outcome of the ADR. In particular, when the frequency of patients with ADR (“positive samples”) are lower than the frequency of patients with no ADR (“negative samples”), the training data instances with positive actual outcomes may be weighted higher than the training data instances with negative actual outcomes. In such a manner, the training process may give more weight to training data instances with positive actual outcomes of ADR.

In one instance, observation weights is given by the vector

$$( \begin{matrix}{r}_{positive} \\ ⋮ \\ {r}_{positive} \\ {r}_{negative} \\ ⋮ \\ {r}_{negative}\end{matrix} )$$

which has length equal to the number of observations (number of patients in a given study) where, rₚₒₛᵢₜᵢᵥₑ is the weight given to the ADR positive outcomes, r_{negative} is the weight given to the ADR negative outcomes. In one instance, the weights for ADR positive outcomes is inversely proportional to the frequency of ADR, such that

$${r}_{p o s i t i v e} = \frac{1}{f}$$

$$where$$

$$f = ( \frac{number of ADR positive patients}{\begin{matrix}total number of study \\ patients in the training data\end{matrix} } )$$

and r_{negative}=1. However, it is appreciated that the values of rₚₒₛᵢₜᵢᵥₑ and r_{negative} can be adjusted depending on the drug, disease, and/or objective of the prediction model. For example, using the observation weights described above, the example loss functions in Equations 8 and 9 can be modified as:

$$( {a}_{t} , {e}_{t} ; w ) = {r}_{p o s i t i v e} \cdot ( t ∈ positive ) \cdot {\sum }_{t = 1}^{T} ( a log {e}_{t} + ( 1 - {a}_{t} ) log ( 1 - {e}_{t} ) )$$

$$( {a}_{t} , {e}_{t} ; w ) = {r}_{p o s i t i v e} \cdot ( t ∈ positive ) \cdot {\sum }_{t = 1}^{T} ( {‖ {y}_{i} - {u}_{i} ‖}_{2}^{2} )$$

where 1(·) is an indicator function, and “positive” indicates the set of training data instances that have positive ADR outcomes.

### 5.9. PREDICTION OF ADVERSE DRUG REACTION

A drug safety score (DSS) described herein can be used to predict drug response of a subject. In some embodiments, the prediction step is performed by the prediction system. In some embodiments, the prediction step is performed by the client device after receiving the drug safety score. In some embodiments, a user performs the prediction step by analyzing the drug safety score based an instruction or a guideline provided with the drug safety score. In some embodiments, a drug safety score is combined with other factors (e.g., environmental factor or other medically relevant information) before being used for prediction of ADRs. The system can also provide a report for the subject indicating a detailed collection of information about the drug safety issues associated with the drug (or for multiple drugs) personalized for that patient. The system can also provide access to a UI that allows a viewer to click through different sets of information about the drug and how it might affect the patient.

In preferred embodiments, a DSS is correlated with a risk of the subject to have an adverse response to the drug. In some embodiments, a low DSS indicates a high risk and a high DSS indicates a low risk, for example, when the actual outcomes in the training data are labeled 0 for positive ADR patients and 1 for negative ADR patients. In some embodiments, a high DSS indicates a high risk and a low DSS indicates a low risk, for example, when the actual outcomes in the training data are labeled 1 for positive ADR patients and 0 for negative ADR patients. In some embodiments, a DSS is a score between 0 and 1 and a DSS close to 0 indicates a low risk to have an adverse drug reaction and a DSS close to 1 indicates a high risk to have an adverse drug reaction. In some embodiments, a DSS is a score between 0 and 1, and a DSS close to 0 indicates a high risk to have an adverse drug reaction and a DSS close to 1 indicates a low risk to have an adverse drug reaction.

DSSs or information related to DSSs can be provided to a user, a subject, a doctor, a pharmacist, or other medical professional. The recipient can use DSSs or related information to understand a phenotype of the patient, for example, to choose a drug or a treatment option, or to derive any other medically relevant information. In some embodiments, a doctor receiving a DSS and/or related information can treat a patient using the information. In some embodiments, a doctor treats a patient with a drug having DSS associated with a low risk. In some embodiments, a doctor treats a patient with a drug having a DSS associated with a lower risk than other drug(s) in the drug group. In some embodiments, a doctor treats a patient with an alternative drug when a drug has a DSS associated with a high ADR risk. In some embodiments, a doctor treats a patient by lowering a drug dose when the drug has a DSS indicating a high ADR risk. In some embodiments, a doctor treats a patient by raising a drug dose when the drug has DSS indicating a low ADR risk. In some embodiments, a doctor monitors a patient more frequently after treatment of the patient with a drug when the drug has a DSS indicating a high ADR risk. In some embodiments, a doctor monitors a patient less frequently after treatment of the patient with a drug when the drug has a DSS indicating a low ADR risk.

DSSs can be further processed and analyzed before being provided to a user, a subject, a doctor, a pharmacist, or other medical professional. DSSs can be calculated with respect all the drugs from which information about one or more associated proteins can be acquired or only some of the drugs. DSSs calculated for multiple drugs can be used to rank the drugs. The ranking can be provided to a user, a subject, a doctor, a pharmacist, or other medical professional for their use. In some embodiments, the ranking can be provided to a doctor to treat a patient based on the ranking. For example, a doctor can treat a patient with a highest-ranked drug or avoid treating a patient with a lowest-ranked drug.

The method of the present disclosure can further include the step of determining the order of priority among drugs applicable to an individual by using the above-described drug safety score; or determining whether to use the drugs applicable to the individual by using the above-described drug safety score. In some embodiments, DSSs can be used to determine optional drug dose for an individual. For example, drug dose can be reduced or raised depending on whether the DSS indicates a high risk or low risk to the drug. When DSS indicates a high risk, the drug dose can be reduced. When DSS indicates a low risk, the drug dose can be raised.

Although DSS can be applied to each of all drugs, it can be more useful when applied to drugs classified by disease, clinical characteristic or activity, or medically comparable drugs. The drug classification system which can be used in the present invention may include, for example, ATC (Anatomical Therapeutic Chemical Classification System) codes, top 15 frequently prescribed drug classes during 2005 to 2008 in the United States (Health, United States, 2011, Centers for Disease Control and Prevention), a list of drugs with known pharmacogenomical markers which can influence the drug effect information described in the drug label, or a list of drugs withdrawn from the market due to side effects thereof. DSS can be compared among drugs in the same drug group.

In some embodiments, DSS of two or more drugs are calculated by methods provided herein when the two or more drugs are to be administered together at the same time or at a short distance of time sufficient to significantly affect pharmacological actions thereof. When two or more drugs need to be administered together, drug safety scores for the two or more drugs can be combined. For example, if two or more drugs do not interact with a same protein, drug safety scores of the two or more drugs can be simply averaged or summed up or multiplied. If there is a protein commonly interacting with the drugs, a protein damage score of the corresponding commonly interacting protein can be assigned with a higher (e.g., double) weighting.

The information related to a combination of two or more drugs can be provided to a doctor or other medical professional to treat a patient with or without the combination. When the combination of multiple drugs has a DSS associated with a high ADR risk, a doctor or other medical professional can treat a patient with a different combination of drugs having a DSS associated with a low ADR risk.

### 5.10. POPULATION DRUG SAFETY SCORE

In some embodiments, a drug safety score (DSS) described herein is calculated for a population of multiple individuals. In some embodiments, a population drug safety score is calculated by using the drug safety scores of multiple individuals.

The term “population drug safety score” as used herein refers to a mean of drug safety scores of individuals belonging to a particular population. The population drug safety score can be obtained by calculating the area under the curve (AUC) of a drug safety score distribution curve, a curve obtained by plotting the drug safety scores of individuals belonging to the population from lower to higher scores, and dividing the AUC by the number of the individuals constituting the population. This is called a standardized area under the curve (S-AUC). When all the drug safety scores in a population are 1, i.e., when there is no variation in drug-related genes which cause functional abnormality of proteins, the area under the curve is equal to the number of the individuals constituting the population. Similarly, the value obtained by dividing the area upper the individual drug safety score distribution curve by the number of the individuals constituting the population is called a standardized area upper the curve (S-AUPC) and it can be used as the population drug safety score. 1-(S-AUPC), which is equal to S-AUC, can also be used as the population drug safety score.

The population drug safety score may be calculated for individual drugs or drug groups considering the characteristics of the drugs. The drug groups may be determined based on known drug classification methods such as the Anatomical Therapeutic Chemical (ACT) Classification System of the WHO, drugs used for identical symptoms, drugs with similar chemical properties, drugs sharing pathways, drugs with identical absorption or excretion mechanisms, drugs with identical targets, etc., although not being limited thereto.

In an exemplary embodiment of the present invention, the population drug safety score (PDSS) is calculated by Equation 10. However, Equation 10 can be modified and the present invention is not limited thereto.

$$PDSS = \frac{1}{N} ( {\sum }_{H = 1}^{N} D S {S}_{d} ) \qquad [ Equation 10 ]$$

In Equation 10, PDSS is a population drug safety score calculated as a mean of drug safety scores of individuals within a population, N is the number of individuals for which the individual drug safety scores, DSSs, are calculated through individual genetic variation analysis. The population may be defined variously based on sex, age, race, disease group, drug medication group, etc., although not being limited thereto. The population drug safety score may be different among different populations.

$$PDSS = \frac{1}{N} ( {AUC}_{d} ) = 1 - \frac{1}{N} ( A U P {C}_{d} ) \qquad [ Equation 11 ]$$

In Equation 11, PDSS is a population drug safety score calculated as a mean of drug safety scores of individuals within a population, AUC_{d} is an area under the individual drug safety score distribution curve for the population, AUPC_{d} is an area upper the individual drug safety score distribution curve for the population and N is the number of individuals for which the individual drug safety scores DSSs are calculated through individual genetic variation analysis. The value obtained by dividing AUC by the number of the individuals belonging to the population is a standardized area under the curve. The value obtained by dividing AUPC by the number of the individuals belonging to the population is a standardized area upper the curve. The population may be defined variously based on sex, age, race, disease group, drug medication group, etc., although not being limited thereto. The population drug safety score may be different among different populations.

The term “drug safety score distribution curve” or “distribution curve of drug safety scores” used in the present invention refers to a plot of the distribution of drug safety scores of individuals within a particular population. It includes a line graph obtained by plotting the drug safety scores from lower to higher scores, a density curve plotted using a density estimation function, a histogram, etc., although not being limited thereto. Further, the population herein may be defined variously based on sex, age, race, disease group, drug medication group, etc., although not being limited thereto. The population drug safety score may be different with respect to different populations and drugs.

In some embodiments, the drug safety threshold score for identifying a high-risk subpopulation is calculated by Equation 12. However, Equation 12 can be modified and the present invention is not limited thereto.

$$T = μ - κ \sqrt{\frac{1}{N} {\sum }_{i = 1}^{N} {( D S {S}_{i} - μ )}^{2}} \qquad [ Equation 12 ]$$

In Equation 12, T is a drug safety threshold score calculated based on S-AUC from the individual drug safety score distribution curve, or an arithmetic mean of individual drug safety scores DSS of a population. T is a rational number satisfying 0<T<1. N is the number of individuals for which the individual drug safety scores DSS are calculated through individual genetic variation analysis, DSSᵢ is a drug safety score of i-th individual and μ is a population drug safety score calculated as an arithmetic mean or a standardized area under the individual drug safety score distribution curve, and κ is an non-zero rational number. When κ is 1, T becomes a score corresponding to the population drug safety score μ subtracted by standard deviation of the individual drug safety scores. When κ is 2, it becomes a score corresponding to the population drug safety score μ subtracted by 2 times of standard deviations of the individual drug safety scores. κ may be varied depending on the distribution of individual drug safety scores within the population. The population may be defined variously based on sex, age, race, disease group, drug medication group, etc., although not being limited thereto. The drug safety threshold score may be different for different populations and drugs.

The term “high-risk subpopulation” used in the present invention refers to a set of individuals having drug safety scores equal to or lower than the drug safety threshold score. It is a subpopulation having many variations causing damage of proteins associated with the pharmacodynamics or pharmacokinetics of the corresponding drug and which is vulnerable to the drug. The drug safety threshold score may be determined based on the pattern of the individual drug safety score distribution curve. That is to say, when there is a subpopulation which forms an island with a remarkably low score distribution in the individual drug safety score distribution curve of the drug, the drug safety threshold score may be calculated as an individual drug safety score defining the island.

R={x|x with DSS<T} [Equation 13]

In Equation 13, R is the ratio or fraction of a high-risk subpopulation with a score lower than the drug safety threshold score in a population, x is an individual with an individual drug safety score (DSS) lower than the drug safety threshold score. The population may be defined variously based on sex, age, race, disease group, drug medication group, etc., although not being limited thereto. The drug safety threshold score may be different for different populations and drugs.

In another exemplary embodiment of the present invention, the threshold score can be estimated through analysis of drug safety scores corresponding to drugs which are withdrawn from the market or whose use has been restricted.

R={x|x with DSS≤T_{w}} [Equation 13]

In Equation 14, R is the ratio or fraction of a high-risk subpopulation with a score lower than the drug safety threshold score in a population, x is an individual with an individual drug safety score lower than the drug safety threshold score and DSS is an individual drug safety score. In some embodiments, T_{w} is 0.3 as calculated based on drugs which are withdrawn from the market or whose use has been restricted. The population may be defined variously based on sex, age, race, disease group, drug medication group, etc., although not being limited thereto. The drug safety threshold score may be different for different populations and drugs and is not limited to 0.3.

Once a high-risk subpopulation is identified, the result can be used by a drug maker, a company running clinical studies, or other pharmaceutical companies in developing a drug, designing clinical studies or selling the drug targeted to a specific population. The result can be also used by physicians when they decide whether to prescribe a certain drug or not. The result can be also used by patients when they decide whether to use a certain drug or not.

In some embodiments, the drug safety scores distribution curve can be used to evaluate safety of drug for a subject. For example, a drug safety score of the subject can be compared with the drug safety scores of multiple individuals within the population or the distribution curve of the scores. If the subject has an individual drug safety score lower than the threshold score described above, or lower than a majority of the individuals in the population, the subject is more likely to have variations that could affect the function of the genes associated with the pharamodynamics and pharmacokinetics of the drug and is more likely to show an undesired side-effect to the drug. Similar analysis can be performed for a number of drugs within a drug group, in order to identify a safest drug to use within the drug group.

Results from the analysis can be provided to the subject or to a physician for the subject. The physician may rely on the results to prescribe the drug, for example, by adjusting a dosage of the drug. Thus, the method of the present invention may be performed for the purpose of preventing side effects of a drug, although not being limited thereto.

### 5.11. EXAMPLES

The following examples are provided by way of illustration not limitation.

### 5.11.1. Example 1: Prediction of Warfarin Adverse Drug Reaction

From the 1990s to 2000s, warfarin was ranked in the top ten drugs with serious side effects, where fifteen to twenty percent of patients suffer from bleeding and one to three percent suffer from intracranial hemorrhage per year. Because of the high prevalence of adverse drug responses and its severity, warfarin is the most studied drug in pharmacogenetics. In the US, 26 commercial tests were conducted and archived in the National Institutes of Health (NIH) genetic testing registry. Analysis results of these tests mainly focused on CYP2C9*2 and CYP2C9*3 and/or VKORC1 (rs9923231) genotypes. As a result, clinicians have conventionally utilized genotyping results of CYP2C9 and VKORC1 in the warfarin dosing calculator to mitigate ADRs from warfarin for a number of years.

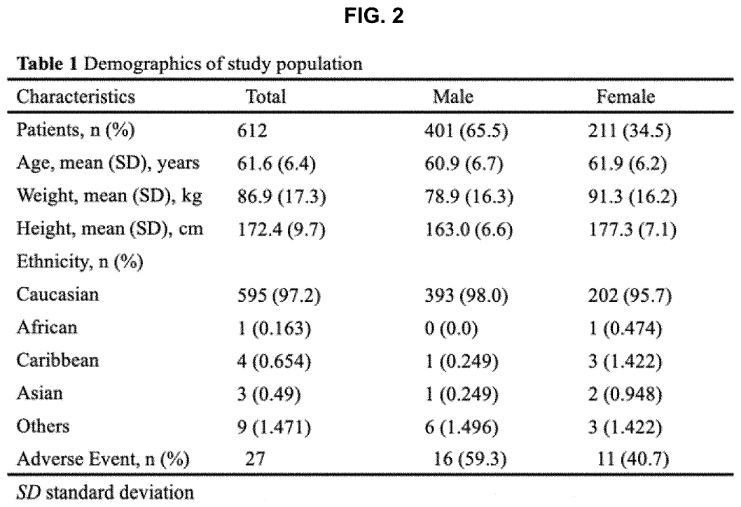

A retrospective analysis was conducted using genomic and phenotypic data from the UK Biobank (FIG. 2). Study inclusion criteria included individuals administered warfarin and having whole exome sequencing (WES) data (n=612). ADR was defined to be positive when the individual had an ADR record (per ICD9/10 codes) within the first 90 days of warfarin administration. Most common warfarin ADRs listed in the health registry data included non-traumatic, hemorrhage, gastrointestinal bleeding, and “ADRs due to anticoagulant use”.

Protein function scores (“PFSs”) of eleven warfarin-associated genes for each individual (ranges from 0 to 1, closer to 0 indicating a higher likelihood of damaged function in gene) was calculated using geometric mean of sequence variation scores of all score-mappable variants within the coding region of respective genes. We then collected demographics and clinical information that are known to be critical for warfarin dosing. They include: weight, height, body mass index (kg/m2), age, sex, ethnicity, amiodarone use (drug-drug-interaction).

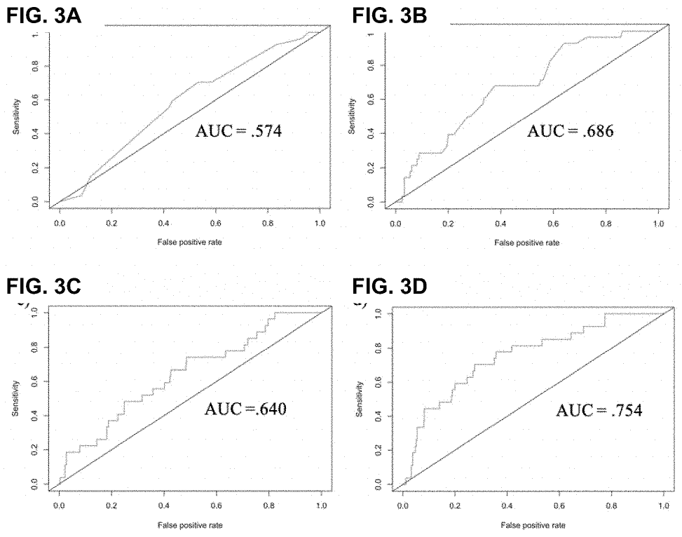

Five-fold cross validated step-wise multiple logistic regression was performed (using observation weight of 20 to 1, giving 20 times higher weight to ADR positive observations) with a hundred repetition, using different combinations of variables for comparison (genotypes of CYP2C9 and VKORC1 only vs. PFS only vs. variables in modified International Warfarin Pharmacogenetics Consortium (IWPC) dosing method only vs. PFS and variables in modified IWPC dosing method). In the experimental results shown in FIGS. 3A-3E, an incoming patient was predicted to suffer ADR to the drug when the predicted DSS of the prediction model for the incoming patient was above a predetermined threshold of 0.5.

A relevant metric indicative of the performance of a prediction model is:

$$Sensitivity = P ( {a}_{v} = 1 \vert D S {S}_{v} \geq threshold ) = \frac{{\sum }_{v ∈ V} ( {a}_{v} = 1 , D S {S}_{v} \geq threshold )}{{\sum }_{v ∈ V} ( DS {S}_{v} \geq threshold )}$$

that indicates the ratio of the number of patients in a validation data set V that were correctly predicted to have an adverse reaction to the drug. As described above, the threshold may be 0.5. Another relevant metric is:

$$\begin{matrix}False positive rate = P ( D S {S}_{v} \geq threshold \vert {a}_{v ∈ V} = 0 ) \\ = \frac{{\sum }_{v ∈ V} ( a = 0 , DS {S}_{v} \geq threshold )}{{\sum }_{v ∈ V} ( {a}_{v} = 0 )} .\end{matrix}$$

As shown in FIGS. 3A-3E, the plots show the relationship between the FPR and the Sensitivity, as well as the area under the curve (AUC). A higher AUC may indicate better performance by the prediction model.

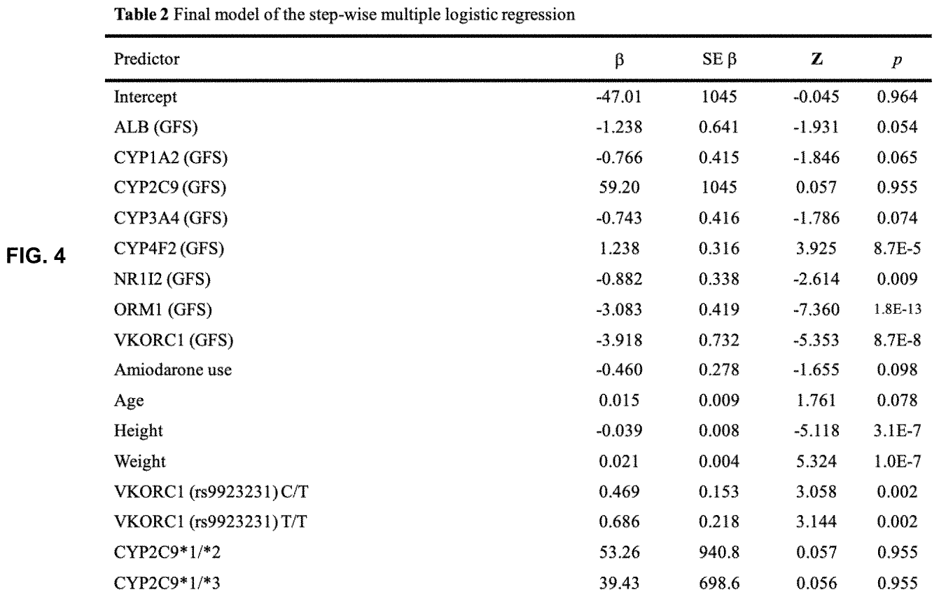

With the final regression model, we analyzed its performance metrics. R and python were used to process all health registry data, and R packages for all statistical tests. Performance of each combination of variables is presented in FIG. 3A-FIG. 3D—genotypes of CYP2C9 and VKORC1 only (FIG. 3A), GFS only (FIG. 3B), variables in modified International Warfarin Pharmacogenetics Consortium (IWPC) dosing method only (FIG. 3C), GFS and variables in modified IWPC dosing method (FIG. 3D). The data showed that adding protein function scores to known variables associated with modified IWPC dosing method yielded the best area under receiver operating characteristics (AUROC) curve (FIG. 3D). Specifically, using the combination of genetic factors and clinical factors resulted in an improvement in the AUC of approximately up to 31% compared to models that only considered genetic factors or only considered clinical factors. Additionally, unlike conventional reliance on CYP2C9 and VKORC1 only for warfarin dosing calculation, GFS of ORM1 and VKORC1 genes had the most significance (β=-3.08 & −3.918, p=1.83×10^{—13} & 8.66×10⁻⁸, respectively) (FIG. 4) among eleven warfarin-associated genes and known variables associated with modified IWPC dosing method. ADR prevalence was 4.41%.

The results in FIG. 3D illustrate the improved predictability of the prediction model described herein, compared to prior techniques of predicting ADR's. Specifically, by applying a machine-learned model, such as a step-wise logistic regression model, the prediction model can easily combine both genetic and clinical factors to predict the likelihood of ADR's, resulting in significant improvement over conventional methods.

Drug ADRs are triggered by an undetermined balance of genetic and environmental factors. It is difficult to quantify the exact impact of genetic variation, as it may account for 20% to 95% of this variability. Protein function score is a tool that is used to elucidate the role of genetics by comprehensively incorporating both rare and common genetic variants in ADR prediction. The regression model based on protein function scores and clinical factor scores generated by the algorithm allows for identification of individuals at higher risk of ADR development.

### 5.11.2. Example 2: Prediction of Chloroquine Adverse Drug Reaction

Chloroquine phosphate is in a class of drugs called antimalarials and amebicides. The drug has been used to prevent and treat malaria and also to treat amebiasis. Chloroquine has been also tested for treatment of coronavirus disease (e.g., COVID-19). Specifically, certain segments of the population are known to have higher risk to COVID-19, including older population with co-morbidities (e.g., cardiovascular disease, hypertension, diabetes), younger population with co-existing disease (e.g., asthma, cardiovascular disease, hypertension, diabetes) and multiple medications, younger population with environmental factors (e.g., smokers). Significant morbidity and mortality of COVID-19 virus are prompting attempts for re-purposing available therapeutics, such as chloroquine and hydroxychloroquine. These are both oral prescription drugs to treat malaria and certain inflammatory conditions such as rheumatoid arthritis, systematic lupus.

Both drugs have in-vitro activity against COVID-19, and based upon limited in-vitro and anecdotal data, chloroquine or hydroxychloroquine are currently recommended for treatment for hospitalized COVID-19 patients in several countries. Both drugs are known to have cadiotoxicity (prolonged QT syndrome of irregular heartbeats) with prolonged use in patients with liver or kidney problems or are immunosuppressed. Optimal dosing and duration of hydroxycologquine for treating COVID-19 are unknown.

Patients with mild or moderate symptoms may benefit from therapeutic treatment, and a patient can progress from a mild/moderate to a severe condition pretty quickly. It is difficult to predict which patient can rapidly turn course, and thus, managing these patients requires quick responses with little room for error and pharmacogenomic testing should be efficient.

Chloroquine has been known to induce adverse drug reactions in some patients. Well known adverse drug reactions include heart problems, changes in your heart rhythm, and hypoglycemia (low blood sugar), which can be life-threatening. In some patients, chloroquine is known to have caused vision problems, extrapyramidal disorders (e.g., dystonia, dyskinesia, tongue protrusion, torticollis), or muscle weakness. Moreover, chloroquine has major drug interactions with other medications (e.g., azithromycin) that can put a person at an even greater risk of an abnormal heart rhythm. Therefore, it is important to predict an adverse drug reaction in a patient before taking the drug.

A retrospective analysis was conducted using genomic and phenotypic data from the UK Biobank. Study inclusion criteria included individuals prescribed with chloroquine or hydrochloroquine and having whole exome sequencing (WES) data (n=333). ADR was defined to be positive when the individual had an ADR record (per ICD9/10 codes) within the prescription window. Most common chloroquine ADRs listed in the health registry data included cardiovascular ADRs, such as cardiac arrhythmia and heart failure.

Protein function scores of six chloroquine-associated genes for each individual (ranges from 0 to 1, closer to 0 indicating a higher likelihood of damaged function in gene) was calculated using geometric mean of sequence variation scores of all score-mappable variants within the coding region of respective genes. The six chloroquine-associated genes include ABCB1, CYP1A1, CYP3A4, CYP3A5, CYP2C8, and CYP2D6. We then collected demographics and clinical information—age, sex, weight and height. We also checked whether each individual was co-prescribed with another drug that is known to interact with chloroquine or hydroxychloroquine and causes or exacerbates long QT syndrome. Such drugs include Macrolides (Azithromycin, erythromycin, etc.), Azoles (voriconazole, itraconazole, etc.), Fluoroquinolones (ciprofloxacin, levofloxacin, etc.).

Five-fold cross validated step-wise multiple logistic regression was performed (using observation weight of 20 to 1, giving 20 times higher weight to ADR positive observations) with a hundred repetition, using different combinations of variables for comparison (demographics and clinical information and co-administered drug vs. co-administered drug and protein function scores of 6 genes vs. demographics and clinical information, co-administered drug, and protein function scores of 6 genes). An incoming patient was predicted to suffer ADR to the drug when the predicted DSS of the prediction model for the incoming patient was above a predetermined threshold of 0.5.

A relevant metric indicative of the performance of a prediction model is:

$$Sensitivity = P ( {a}_{v} = 1 \vert D S {S}_{v} \geq threshold ) = \frac{{\sum }_{v ∈ V} ( {a}_{v} = 1 , D S {S}_{v} \geq threshold )}{{\sum }_{v ∈ V} ( DS {S}_{v} \geq threshold )}$$

that indicates the ratio of the number of patients in a validation data set V that were correctly predicted to have an adverse reaction to the drug. As described above, the threshold may be 0.5. Another relevant metric is:

$$False positive rate = P ( {DSS}_{v} \geq threshold \vert {a}_{v ∈ V} = 0 ) = \frac{{\sum }_{v ∈ V} ( a = 0 , {DSS}_{v} \geq threshold )}{{\sum }_{v ∈ V} ( {a}_{v} = 0 )} .$$

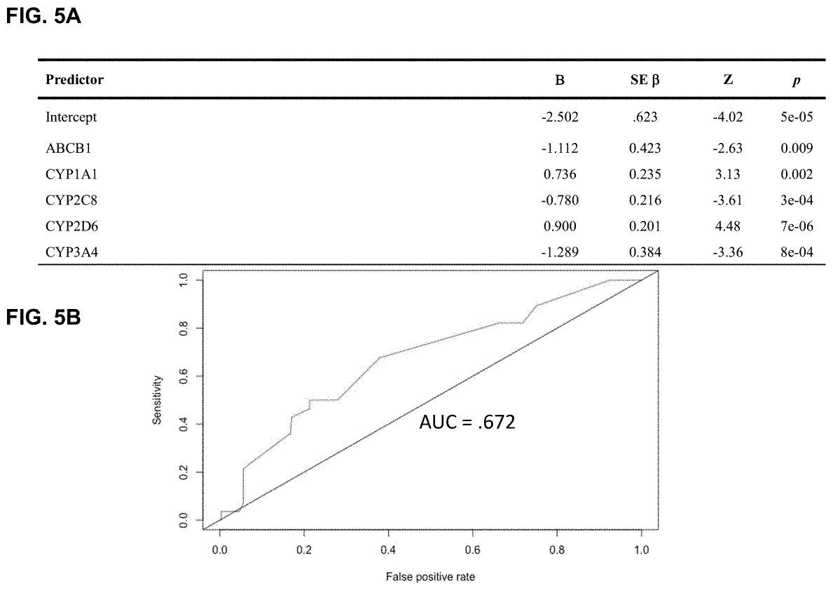

FIG. SA shows the values and statistical characteristics of weightings determined for a stepwise logistic regression model using 6 protein function scores to the study described in Example 2. As shown in FIG. 5, the stepwise logistic regression model includes weightings B each corresponding to a protein function score for one of the 6 chloroquine-associated genes ABCB1, CYP1A1, CYP2C8, CYP2D6, and CYP3A4. “B” denotes the unstandardized value of the weighting for a given factor, “SE B” denotes the standard error for the weighting, “Z” denotes the normalized value of the weighting, and “p” denotes the probability value of the weighting. Also shown in FIG. 5B, the AUC extracted from the false positive rate and sensitivity curve was 0.672.

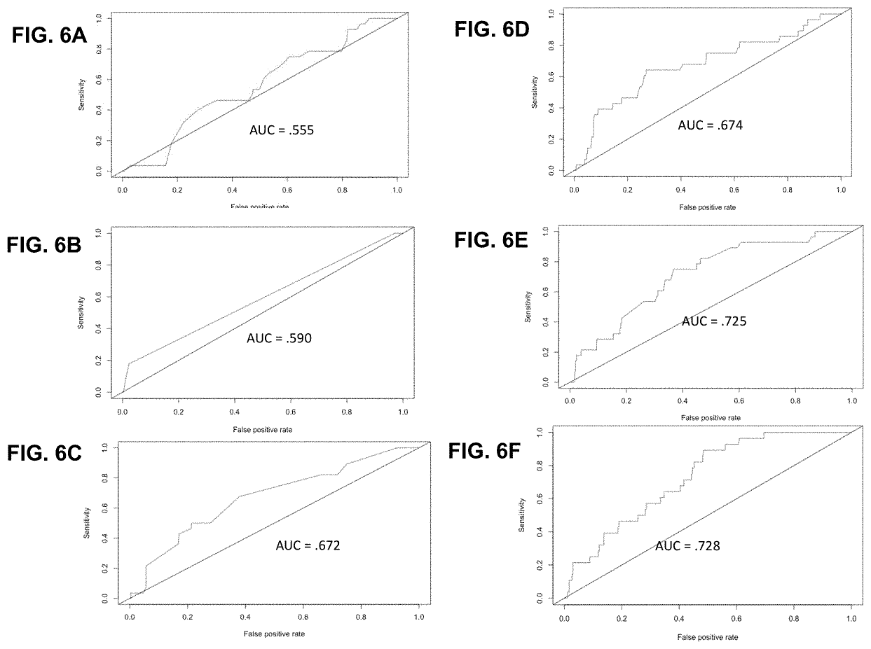

FIG. 6A-6F provide the AUROC curves of step-wise multiple logistic regression models. Specifically, in FIG. 6A, step-wise multiple logistic regression was performed using demographic information, where age was one factor, sex was one factor, weight was one factor, and height was one factor in the prediction model, resulting in an AUC of 0.555. The demographic information can be considered clinical factor scores. In FIG. 6B, step-wise multiple logistic regression was performed using drug-drug-interaction (DDI) factors for Macrolides, Azoles, and Fluoroquinolones as clinical factor scores, resulting in an AUC of 0.590. In FIG. 6C, step-wise multiple logistic regression was performed using protein function scores of the 6 chloroquine-associated genes, resulting in an AUC of 0.672. In FIG. 6D, step-wise multiple logistic regression was performed using the combination of demographic information and the protein function scores of the 6 genes as the factors, resulting in an AUC of 0.674. In FIG. 6E, step-wise multiple logistic regression was performed using the combination of DDI factors and the protein function scores of the 6 genes, resulting in an AUC of 0.725. In FIG. 6F, step-wise multiple logistic regression was performed using the combination of demographic information, DDI factors, and the protein function scores of the 6 genes, resulting in an AUC of 0.728.

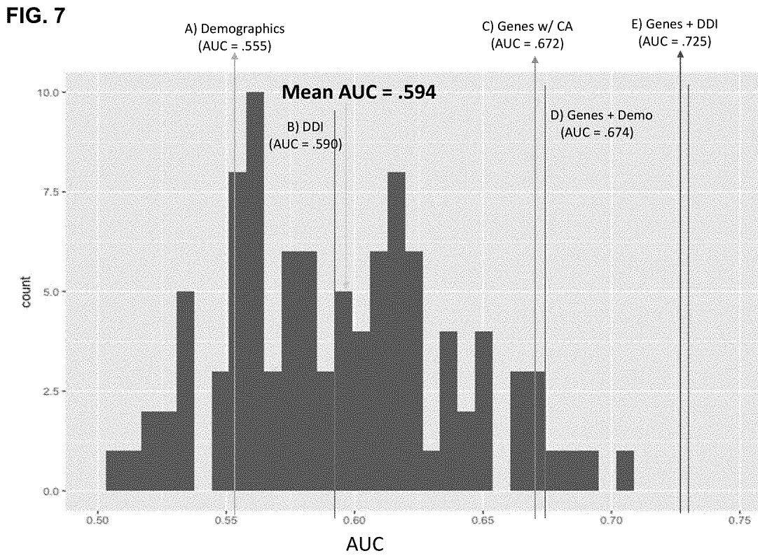

FIG. 7 shows the AUC distribution of prediction models generated by step-wise multiple logistic regression using six random genes. The histogram of the AUCs in solid black bars was obtained by 100 runs of testing using a prediction model trained with the protein function scores of 6 random genes. The mean of AUCs was calculated to be 0.594. The value was compared against AUC values of the prediction models presented in FIG. 6A-E.

As shown in FIG. 7, the comparison indicates that the prediction model using the protein scores of 6 chloroquine-associated genes (AUC=0.672) is significantly better than the prediction model using the protein scores of 6 random genes (AUC=0.594). This demonstrates that the prediction model provided herein performs better than random chances. Use of the protein scores of 6 chloroquine-associated genes together with demographic information (AUC=0.672) or drug-drug interaction (DDI) factors (AUC=0.725) further improved the prediction model.

The data in FIGS. 6A-6F and 7 showed that the prediction model using protein function scores of the 6 chloroquine-associated genes demonstrated statistically significant performance for predicting chloroquine cardiac ADRs. The data also showed that combining clinical factor scores, such as the demographic information and DDI factors, with the protein function scores for chloroquine-associated genes significantly outperformed classical clinical predictions. In particular, the prediction model using protein function scores of the 6 chloroquine-associated genes and DDI factors significantly outperformed the classical clinical prediction models. Thus, the prediction model described herein may be a novel tool that combines the role of genetics (both rare and common genetic variants) and clinical factors such as DDI and demographic information to predict ADR's.

Thus, the prediction model using the machine learning approach described herein provides guided administration of medicines, including chloroquine and hydroxychloroquine, that can help identify high ADR-risk populations and assist physicians in safely prescribing these drugs to COVID-19 patients, and that information can also be used to manage patient post-recovery period.

### 5.11.3. Example 3: Prediction of Direct Oral Anticoagulant (DOAC) Adverse Drug Reaction

Direct oral anticoagulant (DOAC) drugs are a group of anticoagulant medications that either treat or prevents blood clots. DOAC's have been known to induce adverse drug reactions in some patients. Well known adverse drug reactions include gastrointestinal bleeding, non-traumatic hemorrhage, and the like. Therefore, it is important to predict an adverse drug reaction in a patient before taking the drug.

A retrospective analysis was conducted using genomic and phenotypic data from the UK Biobank. Study inclusion criteria included individuals prescribed with DOAC drugs rivaroxaban, dabigaran, or apixaban. ADR was defined to be positive when the individual had an ADR record (per ICD9/10 codes) within the DOAC prescription window.

Relevant genes associated with DOAC's were identified from DrugBank to be ABCB1, ABCG2, ALB, CES1, CES2, CYP1A1, CYP1A2, CYP2C18, CYP2C19, CYP2C8, CYP2C9, CYP2J2, CYP3A4, CYP3A5, CYP3A7, F2, F10, NR1I2, NQO2, ORM1, SULT1A1, UGT1A9, UGT2B7, UGT2B15, VKORC1 from anticoagulation panel. Protein function scores of the identified genes were calculated using geometric mean of sequence variation scores of all score-mappable variants within the coding region of respective genes. Moreover, demographic and clinical information were also collected for the individuals to determine clinical factor scores. The demographic and clinical information included age, weight, height, and sex. The demographic and clinical information also included factors for HASBLED scores. The HASBLED score indicates risk of bleeding for a patient and is generated based on factors such as presence of hypertension, abnormal liver or abnormal liver function, stroke, bleeding, labile INR, elderly age, and/or drug/alcohol use in the patient. The demographic and clinical information also included whether each individual was co-prescribed with another drug that is known to interact with the DOAC's as indicated from the SCVMC protocol (“drug-drug interaction factors” (DDI)).

Five-fold cross validated step-wise multiple logistic regression was performed (using observation weight of 20 to 1, giving 20 times higher weight to ADR positive observations) with a hundred repetition, using different combinations of variables for comparison (demographics and clinical information and co-administered drug vs. co-administered drug and protein function scores of genes vs. demographics and clinical information, co-administered drug, and protein function scores of genes). An incoming patient was predicted to suffer ADR to the drug when the predicted DSS of the prediction model for the incoming patient was above a predetermined threshold of 0.5.

A relevant metric indicative of the performance of a prediction model is:

$$Sensitivity = P ( {a}_{v} = 1 \vert {DSS}_{v} \geq threshold ) = \frac{{\sum }_{v ∈ V} ( a = 0 , {DSS}_{v} \geq threshold )}{{\sum }_{v ∈ V} ( {DSS}_{v} \geq threshold )}$$

that indicates the ratio of the number of patients in a validation data set V that were correctly predicted to have an adverse reaction to the drug. As described above, the threshold may be 0.5. Another relevant metric is:

$$False positive rate = P ( {DSS}_{v} \geq threshold \vert {a}_{v ∈ V} = 0 ) = \frac{{\sum }_{v ∈ V} ( a = 0 , {DSS}_{v} \geq threshold )}{{\sum }_{v ∈ V} ( {a}_{v} = 0 )} .$$

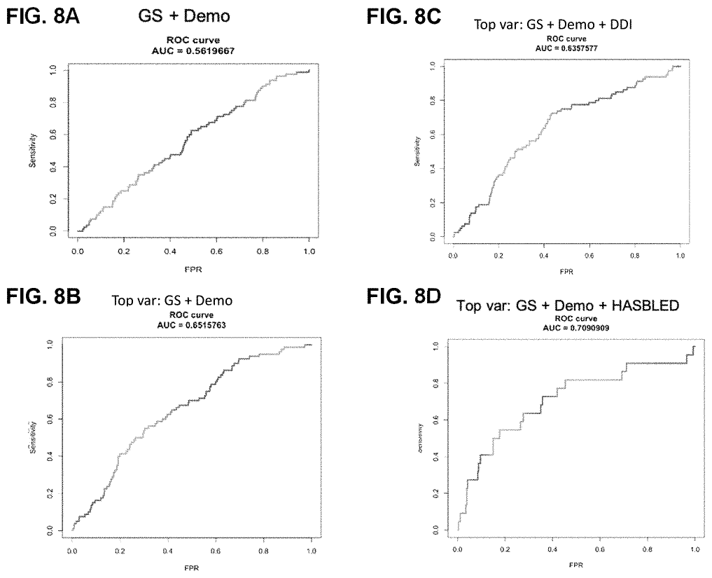

FIG. 8A-8D provide the AUROC curves of step-wise multiple logistic regression models. Specifically, in FIG. 8A, step-wise multiple logistic regression was performed using demographic information, where age was one factor, sex was one factor, weight was one factor, and height was one factor in the prediction model, and protein function scores of the DOAC-related genes, resulting in an AUC of 0.562. The demographic information can be considered clinical factor scores. In FIG. 8B, variables were ranked by their p-values from lowest to highest. Then, step-wise multiple regression was performed using the top n number of variables, and the model with the highest AUC was selected, resulting in an AUC of 0.651. In FIG. 8C, step-wise multiple logistic regression was performed using demographic information and drug-drug-interaction (DDI) factors for the DOAC's for clinical factor scores, and protein function scores for the DOAC-related genes, resulting in an AUC of 0.652. In FIG. 8D, step-wise multiple logistic regression was performed using demographic information and HASBLED factors for clinical factor scores, and protein function scores of the DOAC-related genes, resulting in an AUC of 0.709.

The data in FIGS. 8A-8D showed that the prediction model using protein function scores of the DOAC-related genes demonstrated statistically significant performance for predicting DOAC ADRs. The data also showed that combining clinical factor scores, such as the demographic information, DDI factors, and HASBLED factors, along with the protein function scores for DOAC-associated genes significantly outperformed classical clinical predictions. Thus, the prediction model described herein may be a novel tool that combines the role of genetics (both rare and common genetic variants) and clinical factors such as DDI, demographic information, and HASBLED factors to predict ADR's for DOAC's.

Drug ADRs may be triggered by an undetermined balance of genetic and environmental factors, such as clinical factors. The prediction model using machine learning approach computationally determines an appropriate balance between these factors to improve ADR prediction likelihoods. Moreover, by using machine-learned models, the prediction model can flexibly incorporate a significant number of other genetic or clinical factors that may be helpful for predicting ADRs.

### 6. INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### 7. EQUIVALENTS

While various specific embodiments have been illustrated and described, the above specification is not restrictive. It will be appreciated that various changes can be made without departing from the spirit and scope of the invention(s). Many variations will become apparent to those skilled in the art upon review of this specification.

What is claimed is:

1. A method for treating a subject based on prediction of an adverse reaction to a drug, comprising the steps of:
receiving, by a prediction system, clinical information of the subject related to a plurality of clinical factors (cⱼ);
for each of the clinical factors (cⱼ), determining, by the prediction system, a clinical factor score (S_{cj}) based on the clinical information;
receiving, by the prediction system, individual gene sequence information of the subject;
receiving, by the prediction system, information about a plurality of proteins, wherein each of the proteins is related to pharmacokinetics or pharmacodynamics of the drug;
for each of the genes (gₖ) encoding the proteins,
determining, by the prediction system, a gene sequence variation score (v_{j,k}) for each gene sequence variation of the gene (gₖ) for the subject by using the individual gene sequence information; and
calculating, by the prediction system, an individual protein function score (F_{gk}) associated with the protein by using Equation 2, wherein
Equation 2 is:
$${F}_{gk} ( {v}_{1} , … , {v}_{{n}_{k}} ) = {( {\prod }_{j = 1}^{{n}_{k}} {v}_{j , k}^{{b}_{j , k}} )}^{\frac{1}{{\sum }_{j = 1}^{{n}_{k}} {b}_{j , k}}}$$
wherein F_{gk} is the individual protein function score of the protein encoded by the gene gₖ, nₖ is the number of sequence variations of the gene gₖ, v_{j,k} is a gene sequence variation score of an j^{th} gene sequence variation for the gene gₖ, and b_{j,k} is a weighting assigned to the v_{j,k}; and
determining, by the prediction system, a drug safety score (DSS) by using Equation 7, wherein Equation 7 is:
$$ln ( \frac{DSS}{1 - DSS} ) = {B}_{0} + {W}_{g 1} {F}_{g 1} + {W}_{g 2} {F}_{g 2} + {…W}_{gm} {F}_{gm} + {W}_{c 1} {S}_{c 1} + {W}_{c 2} {S}_{c 2} + {…W}_{cp} {S}_{cp}$$
wherein B₀ is an intercept, W_{gk} is a weighting assigned to each protein function score F_{gk}, W_{ci} is a weighting assigned to each clinical factor score S_{ci}; and
treating the subject with the drug, if the DSS compared to a threshold indicates a low risk of the adverse reaction, and treating the subject with an alternative drug, if the DSS compared to a threshold indicates a high risk of the adverse reaction.

2. The method of claim 1, wherein the weighting (b_{j,k}) assigned to the gene sequence variation score v_{j,k} is determined by:
obtaining training data including a plurality of training instances for a particular protein, each training instance including a predetermined protein function score for the particular protein and a set of gene sequence variation scores for the particular protein,
determining a loss function indicating a difference between the predetermined protein function scores and estimated protein function scores, an estimated protein function score for a training data instance generated by applying Equation 2 to the set of gene sequence variation scores for the training data instance, and
reducing the loss function to determine the weightings assigned to the gene sequence variation scores.

3. The method of claim 1, wherein the weighting (b_{j,k}) assigned to the gene sequence variation score v_{j,k} is 0 for all the gene sequence variation scores.

4. The method of claim 1, wherein the weighting (W_{gk}) assigned to the protein function score F_{gk} and the weighting (W_{ci}) assigned to the clinical function score S_{ci} is determined by:
obtaining training data including a plurality of training instances including information for a plurality of individuals, each training instance including an actual outcome of whether the individual for the training instance experienced an adverse drug reaction and a set of protein function scores and a set of clinical function scores for the individual,
determining a loss function indicating a difference between the actual outcomes and estimated outputs, an estimated output for a training data instance generated by applying Equation 7 to the set of protein function scores and the set of clinical function scores for the training data instance, and
reducing the loss function to determine the weightings assigned to the protein function score and the weightings assigned to the clinical function score.

5. The method of claim 1, wherein the DSS indicates a low risk of the adverse reaction when the DSS is below a threshold.

6. The method of claim 5, wherein the threshold is 0.3, 0.4, or 0.5.

7. The method of claim 1, wherein the clinical factors are selected from the group consisting of age, weight, height, sex, ethnicity, concomitant medication, smoking history, alcohol consumption, and lab data.

8. The method of claim 1, wherein the gene sequence variation score v_{j,k} calculated using one or more algorithms selected from the group consisting of:
SIFT (Sorting Intolerant From Tolerant), PolyPhen (Polymorphism Phenotyping), PolyPhen-2, MAPP (Multivariate Analysis of Protein Polymorphism), Logre (Log R Pfam E-value), MutationAssessor, MutationTaster, MutationTaster2, PROVEAN (Protein Variation Effect Analyzer), PMut, Condel, GERP (Genomic Evolutionary Rate Profiling), GERP++, CEO (Combinatorial Entropy Optimization), SNPeffect, fathmm, CADD (Combined Annotation-Dependent Depletion), and ADME-optimized algorithm.

9. The method of claim 1, wherein the gene sequence variation score v_{j,k} is determined using experimental data.

10. The method of claim 1, wherein the DSS lower than the threshold indicates a low risk of the adverse reaction, and the DSS higher than the threshold indicates a high risk of the adverse reaction.

11. The method of claim 1, wherein the DSS higher than the threshold indicates a low risk of the adverse reaction, and the DSS lower than the threshold indicates a high risk of the adverse reaction.

12. A method for treating a subject based on prediction of an adverse reaction to a drug, comprising the steps of:
receiving, by a prediction system, individual gene sequence information of the subject;
receiving, by the prediction system, information about a protein, wherein the protein is related to pharmacokinetics or pharmacodynamics of the drug, and a gene (g) encoding the protein;
determining, by the prediction system, a gene sequence variation score (v) for each of a gene sequence variation of the gene (g) for the subject by using the individual gene sequence information;
calculating, by the prediction system, an individual protein function score associated with the protein by using Equation 2, wherein
Equation 2 is:
$${F}_{g} ( {v}_{1} , … , {v}_{n} ) = {( {\prod }_{i = 1}^{n} {{v}_{i}}^{{b}_{i}} )}^{\frac{1}{{\sum }_{i = 1}^{n} {b}_{i}}}$$
wherein Fg is the individual protein function score of the protein encoded by the gene g, n is the number of sequence variations of the gene g, vᵢ is a gene sequence variation score of an i^{th} gene sequence variation, and bᵢ is a weighting assigned to the gene sequence variation score vᵢ of the i^{th} gene sequence variation of the gene g, and
wherein the weighting (bᵢ) assigned to the gene sequence variation score vᵢ is determined by:
obtaining training data including a plurality of training instances for a particular protein, each training instance including a predetermined protein function score for the particular protein and a set of gene sequence variation scores for the particular protein,
determining a loss function indicating a difference between the predetermined protein function scores and estimated protein function scores, an estimated protein function score for a training data instance generated by applying Equation 2 to the set of gene sequence variation scores for the training data instance, and
reducing the loss function to determine the weightings assigned to the gene sequence variation scores;
predicting, by the prediction system, likelihood of the adverse reaction to the drug based on the individual protein function score compared to a threshold; and
treating the subject with the drug, if the prediction step indicates low likelihood of the adverse reaction, and treating the subject with an alternative drug, if the prediction step indicates high likelihood of the adverse reaction.

13. The method of claim 12, wherein the gene sequence variation score vᵢ calculated using one or more algorithms selected from the group consisting of:
SIFT (Sorting Intolerant From Tolerant), PolyPhen (Polymorphism Phenotyping), PolyPhen-2, MAPP (Multivariate Analysis of Protein Polymorphism), Logre (Log R Pfam E-value), MutationAssessor, MutationTaster, MutationTaster2, PROVEAN (Protein Variation Effect Analyzer), PMut, Condel, GERP (Genomic Evolutionary Rate Profiling), GERP++, CEO (Combinatorial Entropy Optimization), SNPeffect, fathmm, CADD (Combined Annotation-Dependent Depletion), and ADME-optimized algorithm.

14. The method of claim 12, wherein the gene sequence variation score vᵢ is determined using experimental data.

15. The method of claim 12, further comprising the step of: providing, by the prediction system, the drug safety score (DSS) or information related to the predicted adverse reaction to the drug.

16. The method of claim 15, wherein the DSS lower than a threshold indicates the low likelihood of the adverse reaction, and the DSS higher than the threshold indicates the high likelihood of the adverse reaction.

17. The method of claim 15, wherein the DSS higher than a threshold indicates the low likelihood of the adverse reaction, and the DSS lower than the threshold indicates a high likelihood of the adverse reaction.

18. A system for predicting an adverse drug reaction of a subject to a drug, the system comprising:
a processor;
a computer readable storage medium for storing modules executable by a processor, the modules comprising:
a communication module configured to receive clinical information of the subject related to a plurality of clinical factors (cⱼ), individual gene sequence information for the subject and a plurality of proteins, wherein each of the proteins is related to pharmacokinetics or pharmacodynamics of the drug;
an analysis module configured to:
determine a clinical factor score (Sc_{cj}) for each of the clinical factors (cⱼ),
for each of the genes (gₖ) encoding the proteins, determine a gene sequence variation score (v_{j,k}) for each of a gene sequence variation the gene (gₖ) for the subject by using the individual gene sequence information,
calculate an individual protein function score (F_{gk}) associated with the protein by using Equation 2, wherein
Equation 2 is:
$${F}_{gk} ( {v}_{1} , … , {v}_{{n}_{k}} ) = {( {\prod }_{j = 1}^{{n}_{k}} {v}_{j , k}^{{b}_{j , k}} )}^{\frac{1}{{\sum }_{j = 1}^{{n}_{k}} {b}_{j , k}}}$$
wherein F_{gk} is the individual protein function score of the protein encoded by the gene gₖ, nₖ is the number of sequence variations of the gene gₖ, v_{j,k} is a gene sequence variation score of an j^{th} gene sequence variation for the gene gₖ, and b_{j,k} is a weighting assigned to the v_{j,k},
determine a drug safety score (DSS) by using Equation 7, wherein Equation 7 is:
$$ln ( \frac{DSS}{1 - DSS} ) = {B}_{0} + {W}_{g 1} {F}_{g 1} + {W}_{g 2} {F}_{g 2} + {…W}_{gm} {F}_{gm} + {W}_{c 1} {S}_{c 1} + {W}_{c 2} {S}_{c 2} + {…W}_{cp} {S}_{cp}$$
wherein B₀ is an intercept, W_{gk} is a weighting assigned to each protein function score F_{gk}, W_{ci} is a weighting assigned to each clinical factor score S_{ci}, and
predict the adverse drug reaction of the subject using the drug safety score (DSS); and
an interface generation module configured to provide for display in a user interface on a client device a representation of the prediction for use in treatment of the subject.

19. The system of claim 18, wherein the weighting (b_{j,k}) assigned to the gene sequence variation score v_{j,k} is determined by
obtaining training data including a plurality of training instances for a particular protein, each training instance including a predetermined protein function score for the particular protein and a set of gene sequence variation scores for the particular protein,
determining a loss function indicating a difference between the predetermined protein function scores and estimated protein function scores, an estimated protein function score for a training data instance generated by applying Equation 2 to the set of gene sequence variation scores for the training data instance, and
reducing the loss function to determine the weightings assigned to the gene sequence variation scores.

20. The system of claim 18, wherein the weighting (b_{j,k}) assigned to the gene sequence variation score v_{j,k} is 0 for all the gene sequence variation scores.

21. The system of claim 18, wherein the weighting (W_{gk}) assigned to the protein function score F_{gk} and the weighting (W_{ci}) assigned to the clinical function score S_{ci} is determined by:
obtaining training data including a plurality of training instances including information for a plurality of individuals, each training instance including an actual outcome of whether the individual for the training instance experienced an adverse drug reaction and a set of protein function scores and a set of clinical function scores for the individual,
determining a loss function indicating a difference between the actual outcomes and estimated outputs, an estimated output for a training data instance generated by applying Equation 5 to the set of protein function scores and the set of clinical function scores for the training data instance, and
reducing the loss function to determine the weightings assigned to the protein function score and the weightings assigned to the clinical function score

22. The system of claim 18, wherein the DSS indicates a low likelihood of the adverse reaction when the DSS is below a threshold.

23. The system of claim 22, wherein the threshold is 0.3, 0.4, or 0.5.

24. The system of claim 18, wherein the clinical factors are selected from the group consisting of age, weight, height, sex, ethnicity, concomitant medication, smoking history, alcohol consumption, and lab data.

25. The system of claim 18, wherein the gene sequence variation score v_{j,k} calculated using one or more algorithms selected from the group consisting of:
SIFT (Sorting Intolerant From Tolerant), PolyPhen (Polymorphism Phenotyping), PolyPhen-2, MAPP (Multivariate Analysis of Protein Polymorphism), Logre (Log R Pfam E-value), MutationAssessor, MutationTaster, MutationTaster2, PROVEAN (Protein Variation Effect Analyzer), PMut, Condel, GERP (Genomic Evolutionary Rate Profiling), GERP++, CEO (Combinatorial Entropy Optimization), SNPeffect, fathmm, CADD (Combined Annotation-Dependent Depletion), and ADME-optimized algorithm.

26. The system of claim 18, wherein the gene sequence variation score v_{j,k} is determined using experimental data.

27. A system for predicting an adverse drug reaction of a subject to a drug, the system comprising:
a processor;
a computer readable storage medium for storing modules executable by a processor, the modules comprising:
a communication module configured to receive individual gene sequence information of a subject and information about a protein, wherein the protein is related to pharmacokinetics or pharmacodynamics of the drug, and a gene (g) encoding the protein;
an analysis module configured to:
determine a gene sequence variation score (v) for each of a gene sequence variation of the gene (g) for the subject by using the individual gene sequence information,
calculate an individual protein function score associated with the protein by using Equation 2, wherein
Equation 2 is:
$${F}_{g} ( {v}_{1} , … , {v}_{n} ) = {( {\prod }_{i = 1}^{n} {{v}_{i}}^{{b}_{i}} )}^{\frac{1}{{\sum }_{i = 1}^{n} {b}_{i}}}$$
wherein Fg is the individual protein function score of the protein encoded by the gene g, n is the number of sequence variations of the gene g, vᵢ is a gene sequence variation score of an i^{th} gene sequence variation, and bᵢ is a weighting assigned to the gene sequence variation score vᵢ of the i^{th} gene sequence variation,
wherein the weighting (bᵢ) assigned to the gene sequence variation score vᵢ is determined by:
obtaining training data including a plurality of training instances for a particular protein, each training instance including a predetermined protein function score for the particular protein and a set of gene sequence variation scores for the particular protein,
determining a loss function indicating a difference between the predetermined protein function scores and estimated protein function scores, an estimated protein function score for a training data instance generated by applying Equation 2 to the set of gene sequence variation scores for the training data instance, and
reducing the loss function to determine the weightings assigned to the gene sequence variation scores, and
predict the adverse reaction to the drug based on the individual protein function score; and
an interface generation module configured to provide for display in a user interface on a client device a representation of the prediction for use in treatment of the subject.

28. A computer-readable medium comprising an execution module for executing a processor that performs an operation of predicting an adverse reaction of a subject to a drug, comprising the steps of:
receiving clinical information of the subject related to a plurality of clinical factors (cⱼ);
for each of the clinical factors (cⱼ), determining a clinical factor score (S_{cj}) based on the clinical information;
receiving individual gene sequence information of the subject;
receiving information about a plurality of proteins, wherein each of the proteins is related to pharmacokinetics or pharmacodynamics of the drug;
for each of the genes (gₖ) encoding the proteins,
determining a gene sequence variation score (v_{j,k}) for each of a gene sequence variation of the gene (gₖ) for the subject by using the individual gene sequence information; and
calculating an individual protein function score (F_{gk}) associated with the protein by using Equation 2, wherein
Equation 2 is:
$${F}_{gk} ( {v}_{1} , … , {v}_{{n}_{k}} ) = {( {\prod }_{j = 1}^{{n}_{k}} {v}_{j , k}^{{b}_{j , k}} )}^{\frac{1}{{\sum }_{j = 1}^{{n}_{k}} {b}_{j , k}}}$$
wherein F_{gk} is the individual protein function score of the protein encoded by the gene gₖ, nₖ is the number of sequence variations of the gene gₖ, v_{j,k} is a gene sequence variation score of an j^{th} gene sequence variation for the gene gₖ, and b_{j,k} is a weighting assigned to the v_{j,k}; and
determining a drug safety score (DSS) by using Equation 7, wherein Equation 7 is:
$$ln ( \frac{DSS}{1 - DSS} ) = {B}_{0} + {W}_{g 1} {F}_{g 1} + {W}_{g 2} {F}_{g 2} + {…W}_{gm} {F}_{gm} + {W}_{c 1} {S}_{c 1} + {W}_{c 2} {S}_{c 2} + {…W}_{cp} {S}_{cp}$$
wherein B₀ is an intercept, W_{gk} is a weighting assigned to each protein function score F_{gk}, W_{ci} is a weighting assigned to each clinical factor score S_{ci}; and
predict the adverse drug reaction of the subject using the drug safety score (DSS); and
providing for display in a user interface on a client device a representation of the prediction for use in treatment of the subject.

29. A computer-readable medium comprising an execution module for executing a processor that performs an operation of predicting an adverse reaction of a subject to a drug, comprising the steps of:
receiving individual gene sequence information of the subject;
receiving information about a protein, wherein the protein is related to pharmacokinetics or pharmacodynamics of the drug, and a gene (g) encoding the protein;
determining a gene sequence variation score (v) for each of a gene sequence variation of the gene (g) for the subject by using the individual gene sequence information;
calculating an individual protein function score associated with the protein by using Equation 2, wherein
Equation 2 is:
$${F}_{g} ( {v}_{1} , … , {v}_{n} ) = {( {\prod }_{i = 1}^{n} {{v}_{i}}^{{b}_{i}} )}^{\frac{1}{{\sum }_{i = 1}^{n} {b}_{i}}}$$
wherein Fg is the individual protein function score of the protein encoded by the gene g, n is the number of sequence variations of the gene g, vᵢ, is a gene sequence variation score of an i^{th} gene sequence variation of the gene g, and bᵢ is a weighting assigned to the gene sequence variation score vᵢ of the i^{th} gene sequence variation, and
wherein the weighting (bᵢ) assigned to the gene sequence variation score vᵢ is determined by:
obtaining training data including a plurality of training instances for a particular protein, each training instance including a predetermined protein function score for the particular protein and a set of gene sequence variation scores for the particular protein,
determining a loss function indicating a difference between the predetermined protein function scores and estimated protein function scores, an estimated protein function score for a training data instance generated by applying Equation 2 to the set of gene sequence variation scores for the training data instance, and
reducing the loss function to determine the weightings assigned to the gene sequence variation scores;
predicting, by the prediction system, the adverse reaction to the drug based on the individual protein function score; and
providing for display in a user interface on a client device a representation of the prediction for use in treatment of the subject.

30. A method for selecting a treatment population from a plurality of subjects for treatment with a drug, comprising the steps of:
for each subject in the plurality of subjects:
receiving, by a prediction system, clinical information of the subject related to a plurality of clinical factors (cⱼ);
for each of the clinical factors (cⱼ), determining, by the prediction system, a clinical factor score (S_{cj}) based on the clinical information of the subject;
receiving, by the prediction system, individual gene sequence information of the subject and information about a plurality of proteins, wherein each of the proteins is related to pharmacokinetics or pharmacodynamics of the drug;
for each of the genes (gₖ) encoding the plurality of proteins,
determining, by the prediction system, a gene sequence variation score (v_{j,k}) for each of a gene sequence variation of the gene (gₖ) for the subject by using the individual gene sequence information; and
calculating, by the prediction system, an individual protein function score (F_{gk}) associated with the protein by using Equation 2, wherein
Equation 2 is:
$${F}_{gk} ( {v}_{1} , … , {v}_{{n}_{k}} ) = {( {\prod }_{j = 1}^{{n}_{k}} {v}_{j , k}^{{b}_{j , k}} )}^{\frac{1}{{\sum }_{j = 1}^{{n}_{k}} {b}_{j , k}}}$$
wherein F_{gk} is the individual protein function score of the protein encoded by the gene gₖ, nₖ is the number of sequence variations of the gene gₖ, v_{j,k} is a gene sequence variation score of j^{th} gene sequence variation for the gene gₖ, and b_{j,k} is a weighting assigned to the v_{j,k}; and
determining, by the prediction system, a drug safety score (DSS) for the subject by using Equation 7, wherein Equation 7 is:
$$ln ( \frac{DSS}{1 - DSS} ) = {B}_{0} + {W}_{g 1} {F}_{g 1} + {W}_{g 2} {F}_{g 2} + {…W}_{gm} {F}_{gm} + {W}_{c 1} {S}_{c 1} + {W}_{c 2} {S}_{c 2} + {…W}_{cp} {S}_{cp}$$
wherein B₀ is an intercept, W_{gk} is a weighting assigned to each protein function score F_{gk}, W_{ci} is a weighting assigned to each clinical factor score S_{ci}; and
selecting a treatment population from the plurality of subjects for treatment with the drug based on the determined DSS for the plurality of subjects, the DSS of the selected treatment population indicating a low risk of an adverse reaction to the drug.

31. The method of claim 30, wherein selecting the treatment population from the plurality of subjects comprises selecting the treatment population for a clinical study of the drug.

32. The method of claim 30, wherein the weighting (b_{j,k}) assigned to the gene sequence variation score v_{j,k} is determined by:
obtaining training data including a plurality of training instances for a particular protein, each training instance including a predetermined protein function score for the particular protein and a set of gene sequence variation scores for the particular protein, p1 determining a loss function indicating a difference between the predetermined protein function scores and estimated protein function scores, an estimated protein function score for a training data instance generated by applying Equation 2 to the set of gene sequence variation scores for the training data instance, and
reducing the loss function to determine the weightings assigned to the gene sequence variation scores.

33. The method of claim 30, wherein the weighting (b_{j,k}) assigned to the gene sequence variation score v_{j,k} is 0 for all the gene sequence variation scores.

34. The method of claim 30, wherein the weighting (W_{gk}) assigned to the protein function score F_{gk} and the weighting (W_{ci}) assigned to the clinical function score S_{ci} is determined by:
obtaining training data including a plurality of training instances including information for a plurality of individuals, each training instance including an actual outcome of whether the individual for the training instance experienced an adverse drug reaction and a set of protein function scores and a set of clinical function scores for the individual,
determining a loss function indicating a difference between the actual outcomes and estimated outputs, an estimated output for a training data instance generated by applying Equation 7 to the set of protein function scores and the set of clinical function scores for the training data instance, and
reducing the loss function to determine the weightings assigned to the protein function score and the weightings assigned to the clinical function score.

35. The method of claim 30, wherein the DSS indicates a low risk of the adverse reaction when the DSS is below a threshold.

36. The method of claim 35, wherein the threshold is 0.3, 0.4, or 0.5.

37. The method of claim 30, wherein the clinical factors are selected from the group consisting of age, weight, height, sex, ethnicity, concomitant medication, smoking history, alcohol consumption, and lab data.

38. The method of claim 30, wherein the gene sequence variation score v_{j,k} calculated using one or more algorithms selected from the group consisting of:
SIFT (Sorting Intolerant From Tolerant), PolyPhen (Polymorphism Phenotyping), PolyPhen-2, MAPP (Multivariate Analysis of Protein Polymorphism), Logre (Log R Pfam E-value), MutationAssessor, MutationTaster, MutationTaster2, PROVEAN (Protein Variation Effect Analyzer), PMut, Condel, GERP (Genomic Evolutionary Rate Profiling), GERP++, CEO (Combinatorial Entropy Optimization), SNPeffect, fathmm, CADD (Combined Annotation-Dependent Depletion), and ADME-optimized algorithm.

39. The method of claim 30, wherein the gene sequence variation score v_{j,k} is determined using experimental data.

40. The method of claim 30, further comprising the step of obtaining a curve representing the DSS for the plurality of subjects.

41. The method of claim 30, further comprising the step of determining an area under the curve (AUC), a standardized area under the curve (S-AUC), an area upper the curve (AUPC), or a standardized area upper the curve (S-AUPC).

42. The method of claim 30, further comprising the step of identifying individuals having a DSS below or above a threshold value.

43. The method of claim 42, wherein the threshold value (T) is calculated by the Equation:
$$T = μ - K \sqrt{\frac{1}{n} {\sum }_{i = 1}^{n} {( {DDS}_{i} - μ )}^{2}} ,$$
wherein T is a rational number satisfying 0<T<1, DDSᵢ is an individual drug safety score of an i-th individual (from 1 to n) within the population, n is the number of individuals within the population, κ is a non-zero rational number, and μ is either (i) a mean of the set of individual drug safety scores or (ii) an area under the curve of the set of individual drug safety scores.

44. The method of claim 43, wherein the threshold value (T) is determined based on the shape of the curve.

45. The method of claim 43, wherein the threshold value (T) is calculated based on the change in the slope of the curve.

46. The method of claim 43, wherein the threshold value (T) is determined by comparing the curve with a different curve corresponding to a different drug having similar pharmacodynamics or pharmacokinetics or a different drug previously identified to be unsafe.

47. The method of claim 43, wherein the threshold value (T) ranges from 0.1 to 0.5, from 0.2 to 0.4, or from 0.25 to 0.35, or is 0.3.

48. The method of claims 43, further comprising the step of providing a list of the individuals having a drug safety score below the threshold value or above the threshold value.
